(19) United States
(12) Patent Application Publication
(10) Pub. No.: US 2013/0338457 A1
(43) Pub. Date: Dec. 19, 2013

(54) METHOD AND APPARATUS FOR DETECTING DISEASES ASSOCIATED WITH THE EYE

(71) Applicant: THE REGENTS OF THE UNIVERSITY OF MICHIGAN, Ann Arbor, MI (US)

(72) Inventors: Victor M. Elner, Ann Arbor, MI (US); Howard R. Petty, Livonia, MI (US); Matthew G. Field, Ann Arbor, MI (US)

(73) Assignee: THE REGENTS OF THE UNIVERSITY OF MICHIGAN, Ann Arbor, MI (US)

(21) Appl. No.: 13/901,286

(22) Filed: May 23, 2013

Related U.S. Application Data
(62) Division of application No. 12/270,725, filed on Nov. 13, 2008.
(60) Provisional application No. 60/987,633, filed on Nov. 13, 2007.

(51) Int. Cl. A61B 5/1455; A61B 3/10; A61B 5/00

(52) U.S. Cl. CPC A61B 5/1455; A61B 5/7275; A61B 3/10
USPC 600/317

(57) ABSTRACT

Disease may be detected, monitored, etc. by detecting metabolic dysfunction in a patient's eyes. In one embodiment of an apparatus, an excitation light is generated by an excitation light source to induce autofluorescence in an ocular tissue (e.g., retinal tissue), wherein the excitation light excites flavoprotein autofluorescence (FA) and minimizes the excitation of non-flavoprotein autofluorescence. At least a single image representing the induced ocular tissue autofluorescence is captured. The at least single image is intensified to increase the signal strength of the ocular tissue autofluorescence. The at least single image is analyzed to generate an indicator of whether a patient has one or more of eye damage, a disease that causes eye damage, or to generate an indicator of the progression of a disease, an indicator of the effectiveness of a treatment, a personalized treatment for a subject, etc.

### CROSS-REFERENCE TO RELATED APPLICATIONS

This application claims the benefit of priority under 35 U.S.C. §120 of U.S. patent application Ser. No. 12/270,725, filed Nov. 13, 2008, and under 35 U.S.C. §119(e) of U.S. Provisional Patent Application No. 60/987,633, filed Nov. 13, 2007, both of which are incorporated herein by reference in their entireties and for all purposes.

### STATEMENT REGARDING FEDERAL RESEARCH

This invention was made with government support under CA074120, EY007003 and EY009441, awarded by the National Institutes of Health. The government has certain rights in the invention.

### FIELD OF THE DISCLOSURE

The present disclosure relates generally to medical devices and diagnostic methods and, more particularly, to medical devices and methods used for detecting systemic and optic conditions associated with apoptosis.

### BACKGROUND

Failure of any retinal component can result in blindness. For example, total or partial blindness can be caused by a reduction in blood supply to the retina, which in turn, can be the result of diabetic retinopathy or ischemic events such as retinal vein occlusion. Research has shown that other causes of blindness such as cytomegalovirus retinitis, glaucoma, Leber's optic neuropathy, retina detachment, age-related macular degeneration, retinitis pigmentosa, or light induced blindness are commonly associated with the apoptotic, or programmed death of retina cells.

Mitochondria are critical cell organelles whose primary function is to provide energy, in the form of adenosine triphosphate (ATP), to power essential cellular processes. Mitochondria are also recognized to play a crucial role in the process of programmed cell death or apoptosis.

Apoptosis generally involves the activation of one or more apoptotic signaling pathways by intrinsic or extrinsic stimuli causing the selective degeneration of neurons. Apoptosis occurs via a careful interplay of mitochondrial membrane permeability, which results in uncoupling of the respiratory chain, cessation of ATP production, induction of the apoptotic cascade and ultimately cell death. The onset of apoptosis has been linked to mitochondrial dysfunction (which is indicative of a change in cellular metabolic activity) characterized by the loss of mitochondrial integrity leading to the release of apoptotic mediators and the activation of enzymes and other pathways leading to cell death. These changes in mitochondrial integrity have been linked to the retina disorders that result in 95% of the instances of irreversible blindness. Early detection of mitochondrial dysfunction can allow for diagnosis, treatment, and monitoring of these disorders. Pre-apoptotic cells or those with stressed mitochondria develop impaired electron transport by the energy-generating enzymes in the respiratory chain and other metabolic pathways, which cause increased percentages of several flavoproteins (FP) to become oxidized. These oxidized or electron-poor FP are capable of absorbing blue light and emitting green autofluorescence.

Flavoprotein autofluorescence (FA) has been recognized as a measure of mitochondrial dysfunction or cell stress in non-ocular tissues. Skeletal muscle, liver, and heart muscle were among the first tissues in which ex vivo FP was studied due to these tissues having high metabolic rates and greater numbers of mitochondria. Subsequent studies in vivo have indicated that FA is elevated in apoptosis-prone regions of ischemia-reperfusion injury in heart and brain tissue and in chondrocytes prone to apoptosis. FA elevations in these studies correlated well with other markers of apoptosis such as Bcl-2 depletion and mitochondrial transmembrane potential (φ) instability. Flow cytometric studies have also been adapted to examine FA to detect mononuclear cell mitochondrial dysfunction in patients with chronic progressive external ophthalmoplegia. Thus, in tissues outside of the eye, detection of FA is well accepted as a key method in assessing mitochondrial health.

Current diagnostic techniques used in routine eye examinations typically employ ophthalmoscopes to visually inspect the retina and tonometers to evaluate intraocular pressures. While ophthalmoscopes can be used to diagnose retinal degeneration, they are only effective after substantial damage has already occurred and do not provide any indication of mitochondrial activity. Tonometers indent the eye in order to determine changes in intraocular pressure that can result in glaucoma, retinal ganglion cell death, or ischemia. However, the correlation between intraocular pressure and disease is not robust, as evidenced by patients developing glaucomatous degeneration with low pressures and patients with high pressure remaining disease free. Furthermore, these older methods cannot be correctly interpreted in the presence of biomechanical artifacts such as abnormal corneal thickness due to, for example, natural variations, disease, myopia, or refractive corneal surgery.

Hyperglycemia induces mitochondrial stress and apoptotic cell death in diabetic tissues soon after disease onset and before involvement can be detected by any current clinical diagnostic method. It would be advantageous to measure mitochondrial metabolic activity for an early indicator of the onset of disease. The gold standard diagnostic method for diabetes is the oral glucose tolerance test. However, this method is cumbersome and is often avoided by patients. Thus, many diabetics can remain undiagnosed until they develop diabetic micro- and macrovascular complications.

U.S. Pat. No. 4,569,354 entitled “Method and Apparatus for Measuring the Natural Retinal Fluorescence” discloses a method and apparatus for determining oxygenation of the retina by measuring the fluorescence of flavoprotein in the retina. According to this patent, a spot of excitation light of a wavelength of about 450 nanometers (nm) is scanned across the retina, in response to which retina autofluorescence at a wavelength of about 520 nm is detected. In particular, retinal emission light is detected at two wavelengths of about 520 nm and 540 nm to allow for compensation with respect to absorption and transmission variables in the eye. To compensate for fluorescence of the lens of the eye, the center of the pupil is imaged onto scanning mirrors so that the scanning beam of excitation light pivots at the center of the eye lens. Because this method and apparatus scans a small area of the retina (i.e. a very limited number of pixels) at a time, the strength of the measured signal is extremely low, resulting in a measured signal having a low signal-to-noise (S/N) ratio and little, if any, accuracy. Further, the small scan area necessitates an extended procedure time to completely scan the retina, which further increases potential for error caused by eye movement due to natural instability of extraocular muscle tone, blood pulsation and light contamination. Because of the inherent inaccuracies of this method and device, it is unable to operate as an accurate diagnosis and monitoring system.

U.S. patent application Ser. No. 10/777,423 to Petty et al., filed Feb. 12, 2004, generally describes devices for measuring auto-fluorescence of a retina to measure apoptotic activity of an eye. In one embodiment, an auto-fluorescence measurement device includes an excitation light source and an image capture device. In this embodiment, the image capture device records a single image representative of a retinal fluorescence signal generated immediately in response to the excitation light. In this embodiment, a filter maximizes the passage of auto-fluorescence and an image intensifier provides a focused amplified image showing evidence of apoptotic activity in the eye.

There is a need for a device and method that can provide for accurate and early diagnosis of various systemic diseases such as diabetes based on metabolic dysfunction that are discernable in the eyes.

### SUMMARY

In one embodiment, an apparatus includes an excitation light source adapted to excite flavoprotein autofluorescence while minimizing the excitation of non-flavoprotein autofluorescence, an image capture device adapted to record a single image representative of an ocular tissue (e.g., retinal tissue) fluorescence signal generated in response to the excitation light. The image capture device includes a filter adapted to minimize attenuation of flavoprotein autofluorescence while attenuating non-flavoprotein autofluorescence, and an image intensifier adapted to increase the ocular tissue fluorescence signal strength. The apparatus further includes a computing device communicatively coupled to the image capture device, the computing device configured to generate an indication of an intensity variance for the single image.

In another embodiment, a method includes providing an excitation light generated by an excitation light source to induce autofluorescence in an ocular tissue (e.g., retinal tissue), wherein the excitation light excites flavoprotein autofluorescence and minimizes the excitation of non-flavoprotein autofluorescence, and capturing a single image representing the induced ocular tissue autofluorescence. The method also includes intensifying the single image to increase the signal strength of the ocular tissue autofluorescence, and analyzing the single image to determine an intensity variance for the single image.

In yet another embodiment, an apparatus includes an excitation light source adapted to excite flavoprotein autofluorescence while minimizing the excitation of non-flavoprotein autofluorescence, a filter adapted to minimize attenuation of flavoprotein autofluorescence while attenuating non-flavoprotein autofluorescence; and a photo detector coupled to the filter to detect an ocular tissue (e.g., retinal tissue) fluorescence signal generated in response to the excitation light and to generate a signal indicative of an integrated intensity of the ocular tissue fluorescence signal, The apparatus also includes a photon intensifier coupled to the photo detector to increase the ocular tissue fluorescence signal, and a computing device communicatively coupled to the photo detector, the computing device configured to generate, based on the signal indicative of the integrated intensity, one or more of an indication of a degree of ocular tissue damage, an indication of a degree of ocular tissue distress, an indication of whether a patient has diabetes (e.g., overt diabetes, pre-diabetes, gestational diabetes, etc.), an indication of whether the patient has an eye condition caused by diabetes, an indication of whether a patient has central serous retinopathy, an indication of whether the patient has diabetic retinopathy, an indication of whether the patient has retinal vascular occlusion, an indication of whether the patient has vitreoretinopathy, an indication of whether the patient has retinal vascular disease, an indication of whether the patient has infectious and/or non-infectious uveitis and/or retinitis, an indication of whether the patient has any other acquired retinopathy, an indication of whether the patient has age-related macular degeneration, an indication of whether the patient has inherited retinal degeneration, an indication of whether the patient has pseudotumor cerebri, an indication of whether the patient has glaucoma, an indication of whether the patient has thyroid eye disease, an indication of whether the patient has optic neuritis, an indication of whether the patient has Graves disease, and an indication of whether a patient has an optic nerve condition.

In still another embodiment, a method includes providing an excitation light generated by an excitation light source to induce autofluorescence in an ocular tissue (e.g., retinal tissue), wherein the excitation light excites flavoprotein autofluorescence and minimizes the excitation of non-flavoprotein autofluorescence. Additionally, the method includes detecting an induced ocular tissue autofluorescence signal, intensifying the ocular tissue autofluorescence signal, and analyzing the ocular tissue autofluorescence signal to generate one or more of an indication of a degree of ocular tissue damage, an indication of a degree of ocular tissue distress, an indication of whether a patient has diabetes (e.g., overt diabetes, pre-diabetes, gestational diabetes, etc.), an indication of whether the patient has an eye condition caused by diabetes, an indication of whether the patient has central serous retinopathy, an indication of whether the patient has diabetic retinopathy, an indication of whether the patient has retinal vascular occlusion, an indication of whether the patient has vitreoretinopathy, an indication of whether the patient has retinal vascular disease, an indication of whether the patient has infectious and/or non-infectious uveitis and/or retinitis, an indication of whether the patient has any other acquired retinopathy, an indication of whether the patient has age-related macular degeneration, an indication of whether the patient has inherited retinal degeneration, an indication of whether the patient has pseudotumor cerebri, an indication of whether the patient has glaucoma, an indication of whether the patient has thyroid eye disease, an indication of whether the patient has optic neuritis, an indication of whether the patient has Graves disease, and an indication of whether a patient has an optic nerve condition.

In yet another embodiment, an apparatus includes an excitation light source adapted to excite flavoprotein autofluorescence while minimizing the excitation of non-flavoprotein autofluorescence, and an image capture device adapted to record a single image representative of an ocular tissue (e.g., retinal tissue) fluorescence signal generated in response to the excitation light. The image capture device includes a filter adapted to minimize attenuation of flavoprotein autofluorescence while attenuating non-flavoprotein autofluorescence, and an image intensifier adapted to increase the ocular tissue fluorescence signal strength. The apparatus further includes a computing device communicatively coupled to the image capture device, the computing device configured to generate one or both of an indication of whether a patient has diabetes (e.g., overt diabetes, pre-diabetes, gestational diabetes, etc.) and an indication of whether a patient has an eye condition caused by diabetes.

In another embodiment, a method includes providing an excitation light generated by an excitation light source to induce autofluorescence in an ocular tissue (e.g., retinal tissue), wherein the excitation light excites flavoprotein autofluorescence and minimizes the excitation of non-flavoprotein autofluorescence. The method also includes capturing a single image representing the induced ocular tissue autofluorescence, intensifying the single image to increase the signal strength of the ocular tissue autofluorescence, and analyzing the single image to generate one or both of indicator of whether a patient has diabetes (e.g., overt diabetes, pre-diabetes, gestational diabetes, etc.) and an indicator of whether a patient has an eye condition caused by diabetes.

In still another embodiment, a method includes providing an excitation light generated by an excitation light source to induce autofluorescence in an ocular tissue (e.g., retinal tissue), wherein the excitation light excites flavoprotein autofluorescence and minimizes the excitation of non-flavoprotein autofluorescence. The method also includes capturing a single image representing the induced ocular tissue autofluorescence, intensifying the single image to increase the signal strength of the ocular tissue autofluorescence, and analyzing the single image to generate an indicator of whether a patient has an optic nerve condition.

In yet another embodiment, an apparatus includes an excitation light source adapted to excite flavoprotein autofluorescence while minimizing the excitation of non-flavoprotein autofluorescence, and an image capture device adapted to record a single image representative of an ocular tissue (e.g., retinal tissue) signal generated in response to the excitation light. The image capture device includes a filter adapted to minimize attenuation of flavoprotein autofluorescence while attenuating non-flavoprotein autofluorescence, and an image intensifier adapted to increase the ocular tissue fluorescence signal strength, The apparatus further includes a computing device communicatively coupled to the image capture device, the computing device configured to generate one or more of an indication of whether a patient has central serous retinopathy, an indication of whether the patient has diabetic retinopathy, an indication of whether the patient has retinal vascular occlusion, an indication of whether the patient has vitreoretinopathy, an indication of whether the patient has retinal vascular disease, an indication of whether the patient has infectious and/or non-infectious uveitis and/or retinitis, an indication of whether the patient has any other acquired retinopathy, an indication of whether the patient has age-related macular degeneration, an indication of whether the patient has inherited retinal degeneration, an indication of whether the patient has pseudotumor cerebri, an indication of whether the patient has glaucoma, an indication of whether the patient has thyroid eye disease, an indication of whether the patient has optic neuritis, and an indication of whether the patient has Graves disease.

In another embodiment, a method includes providing an excitation light generated by an excitation light source to induce autofluorescence in an ocular tissue (e.g., retinal tissue), wherein the excitation light excites flavoprotein autofluorescence and minimizes the excitation of non-flavoprotein autofluorescence. The method also includes capturing a single image representing the induced ocular tissue autofluorescence, intensifying the single image to increase the signal strength of the ocular tissue autofluorescence, and analyzing the single image to generate one or more of an indicator of whether a patient has central serous retinopathy, an indicator of whether the patient has diabetic an indicator of whether the patient has retinopathy, an indicator of whether the patient has retinal vascular occlusion, an indicator of whether the patient has vitreoretinopathy, an indicator of whether the patient has retinal vascular disease, an indicator of whether the patient has infectious and/or non-infectious uveitis and/or retinitis, an indicator of whether the patient has any other acquired retinopathy, an indicator of whether the patient has age-related macular degeneration, an indicator of whether the patient has inherited retinal degeneration, an indicator of whether the patient has pseudotumor cerebri, an indicator of whether the patient has glaucoma, an indicator of whether the patient has thyroid eye disease, an indicator of whether the patient has optic neuritis, and an indicator of whether the patient has Graves disease.

Additionally, the present disclosure describes methods of detecting disease by detecting metabolic and/or mitochondrial dysfunction in a subject's eyes.

Further, the present disclosure also describes methods of early detection of disease by detecting modified flavoprotein autofluorescence (FA) in mitochondria of a subject's eyes before otherwise detectable clinical symptoms.

Also, the present disclosure describes methods of prescreening for disease by detecting FA in mitochondria of a subject's eyes before detecting clinical symptoms of a disease, and based on results of the detecting step, recommending and/or performing further clinical testing.

Additionally, the present disclosure describes methods of monitoring disease progression by detecting FA in a subject's eyes at a first time point, detecting FA from at least a second time point, comparing the FA at the first and second time point, and determining the progression of disease.

Further, the present disclosure describes methods of detecting ocular changes due to an effect of a substance by detecting modified FA in mitochondria of a subject's eyes while a substance is known to be present in the subject.

The present disclosure describes methods of testing treatments on a subject for effectiveness by administering at least one treatment to a subject with a disease, detecting FA in a subject's eyes at a first time point, detecting FA at at least a second time point, comparing the FA at the first and second time point, and determining the effectiveness of the treatment on disease.

The present disclosure also describes methods of personalized medicine by determining an effective treatment for a particular subject as above, and administering the treatment to the subject.

The present disclosure additionally describes apparatus comprising a mechanism for detecting systemic disease by detecting metabolic and/or mitochondrial dysfunction in a subject's eyes.

### BRIEF DESCRIPTION OF THE DRAWINGS

Other advantages that may be provided by one or more (or none) of various embodiments of the present invention will be readily appreciated as the same becomes better understood by reference to the following detailed description when considered in connection with the accompanying drawings wherein:

FIG. 1 is a block diagram of one embodiment of an apparatus according to the present invention;

FIG. 2 is a flow diagram of an example method of generating an indicator of one or more of eye damage, a disease of the eye, a disease that causes damage to the eye;

FIG. 3 is a block diagram of an example computing device;

FIG. 4 is a chart of clinical and retinal flavoprotein autofluorescence (FA) data for 6 patients with pseudotumor cerebri;

FIGS. 5A-5C are retinal FA histograms of pixel intensities collected from three age-matched volunteers;

FIG. 6 is a graph of average intensity (AI) of 20 out of 21 diabetics from FIG. 2 with (▪) retinopathy in at least one eye and without (□) retinopathy in either eye compared to their concurrent HbA1c (One unavailable);

FIG. 7 is a table showing mean AI and average curve width (ACW) levels in diabetics and controls by age category;

FIG. 8 is a graph of number of pixels versus autofluorescence intensity for a normal eye and an eye with disease;

FIG. 9A is a retinal photograph and FIG. 9B is a conventional fluorescein angiography of a left eye, and FIG. 9C is a graph of FA curves;

FIG. 10 is a graph of AI of retinal FA from 21 diabetics (with (▪) or without (□) retinopathy) and 21 age-matched controls; for each volunteer, values for the right and left eyes are shown paired; pairs are segregated by vertical gridlines;

FIG. 11 is a graph of ACW from 21 diabetics with (▪) or without (□) retinopathy and 21 age-matched controls; for each volunteer, values for the right and left eyes are shown paired; pairs are segregated by vertical gridlines;

FIG. 12 is a bar graph of average pixel intensities (AI) for retinal FA from control volunteers in three age groupings;

FIGS. 13A-13D are histograms of retinal FA pixel intensities from four age-matched patients demonstrating increased pixel intensity in diabetes mellitus and particularly in diabetic retinopathy, right eye=blue, left eye=red;

FIG. 14 us a bar graph of average pixel intensities from patients with diabetic retinopathy (ages 56-68, n=7), diabetics without retinopathy (ages 54-67, n=7), and age-matched non-diabetic controls (ages 57-67, n=7);

FIGS. 15A-15B are retinal FA pixel intensity histograms from a 77 year old patient with ARMD (16A) and a 67 year old patient without visible clinical findings of ARMD (16B);

FIGS. 16A-16B are FA pixel histograms from a 49 year old patient with bilateral central serous retinopathy (17A) and an age-matched control (17B);

FIGS. 17A-17B are FA pixel intensity histograms from a 36 year old patient with retinitis pigmentosa (18A) and an age-matched control (18B);

FIGS. 18A-18B are bar graphs of FA production induced by hydrogen peroxide (18A) and C2-ceramide (18B) in human retinal pigment epithelium (RPE) cells;

FIGS. 19A, 19C, and 19E are fundus photographs of the affected eyes, and FIGS. 19B, 19D, and 19F are flavoprotein fluorescence (FPF) histograms of the affected and unaffected eyes (right, black; left, grey) of three patients with unilateral central serous retinopathy (CSR);

FIG. 20 is a bar graph of retinal FPF AI of the affected and unaffected eyes of three patients with unilateral CSR and the average retinal FPF AI of control volunteers, age-matched control FPF AI for each patient was obtained from six eyes of three volunteers, all within 1-2 years of the patient's age;

FIG. 21 is a graph of FA data from a control patient, the intensities and curve widths of FA of both eyes are similar, right eye=blue; left eye=red;

FIG. 22 is a graph of FA data from a 63 year old male glaucoma patient with no co-existing ocular or systemic disease that might influence the findings, the left eye (red) is affected by disease, further illustrated by the visual fields in FIG. 24;

FIG. 23 is a computerized visual field of a patient whose FA findings are shown in FIG. 22, an early, subtle visual field defect is in the left eye and the right field is full;

FIG. 24 is a bar graph of in vitro determination of cytoplasmic histone-associated DNA fragments (mono- and oligonucleosomes) after induced cell death;

FIG. 25 is a bar graph of FA production induced by hydrogen peroxide in human retina cells and attenuated with the presence of the oxidation inhibitor N-acetyl-cysteine;

FIG. 26 is a bar graph of FA production induced by hydrogen peroxide in rat neural retina cells and attenuated with the presence of the oxidation inhibitor N-acetyl-cysteine; and

FIGS. 27A-27D are bar graphs of FA of human RPE cells incubated with apoptotic stimuli (hydrogen peroxide and C2-ceramide) and the effects of blocking mitochondrial metabolism and flavoprotein expression on the autofluorescence signal.

### DETAILED DESCRIPTION

In at least some embodiments according to the present invention, the presence of disease in a patient is determined based on measuring flavoprotein autofluorescence (FA) in a patient's retina or some other ocular tissue or tissue associated with the eye.

Hyperglycemia induces mitochondrial stress and apoptotic cell death in diabetic tissues soon after disease onset and before involvement can be detected by any current clinical diagnostic method. Thus, the measurement of mitochondrial metabolic activity can serve as an early indicator of the onset of disease. Prior to apoptosis, mitochondria exhibit impaired electron transport by energy-generating enzymes in the respiratory chain, causing increased percentages of flavoproteins in the chain to be oxidized and rendered capable of absorbing blue light and emitting green autofluorescence. Thus, increased flavoprotein autofluorescence can be an early indicator of diabetic metabolic tissue stress.

The word “image” as used herein, refers to an actual image taken with a device such as example devices described herein. In describing an example apparatus below, reference to a “single” image is generally used; however, multiple single images can also be taken or utilized in each embodiment. Photometric readings can also be taken with a devices such as the example devices described herein, and such readings can be taken wherever an image is taken, for example.

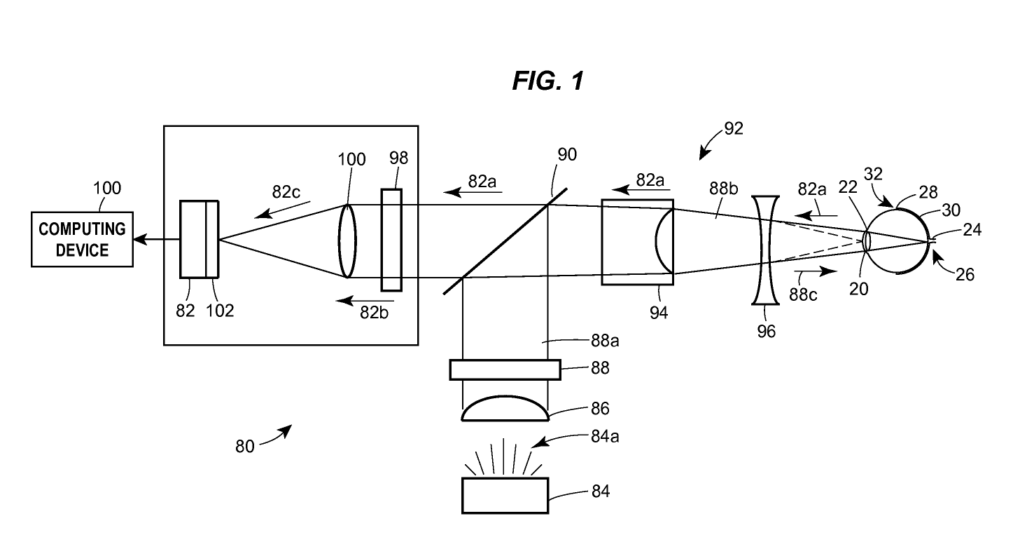

FIG. 1 is a block diagram of an example apparatus 80 that may be utilized for detecting eye damage (e.g., retinal damage, optic nerve damage, etc.) caused by diabetes (e.g., overt diabetes, pre-diabetes, gestational diabetes, etc.). As explained in more detail below, the apparatus 80 additionally or alternatively can be utilized for helping to determine whether a patient has diabetes (e.g., overt diabetes, pre-diabetes, gestational diabetes, etc.). For instance, detected eye damage can be an indicator of diabetes.

The apparatus 80 includes an image capture device 81 and an excitation light source 84. Generally speaking, the light source 84 generates light that excites FA in ocular tissue such as a retina 30, and the image capture device 81 captures an image of the FA signal from the retina 30. The image capture device 81 can include a camera, such as a charge-coupled device (CCD) camera. If a CCD camera is used, it can be, for example, a cooled CCD camera that can include a Peltier cooler to reduce the temperature of the detector and thereby decrease thermally generated electronic noise or dark current noise. It will be understood that the camera can be selected to have a field of view (FOV) optimized to capture the single image of the FA from the retina 30. The apparatus 80 can be configured so that a flash of light from the light source causes the FA from the retina 30, and the image capture device 81 captures the single image of the retinal FA. The single image can be analyzed in a manner that indicates metabolic activity and/or health of the subject retina 30, and thus a direct and non-invasive procedure is provided.

In operation, the excitation light source 84 cooperates with a focusing lens 86 to direct the emitted excitation light 84a to an excitation filter 88. The excitation light source 84 can be He—Cd or argon-ion laser, an incandescent or mercury lamp such as an ATTOARC™ variable intensity illuminator, a light emitting diode (LED), etc. The excitation filter 88 can be, for example, a passband filter having a passband located at approximately 467 nm (such as provided by OMEGA OPTICAL®). The excitation filter 88 can be selected to attenuate wavelengths that do not correspond to the excitation wavelength of FA (e.g., wavelengths of or around 467 nm). The filtered light 88a can then be directed to a dichroic reflector 90, such as a 495 nm long-pass dichroic reflector, for redirection towards the subject retina 30.

Generally speaking, it is desirable to reduce potential signal noise by limiting the excitation spectrum to a range consistent with the excitation spectrum of FP, approximately 467 nm. To this end, an excitation filter such as described above can be used. The filtered excitation means stimulates the FP autofluorescence without stimulating additional molecules and thereby generating unwanted autofluorescence that could act as noise to degrade the overall accuracy of the evaluation technique. It will be understood that the excitation spectrum can be further limited by reducing the ambient light adjacent to the retina 30, which can be accomplished by reducing the testing room lighting, by fitting the subject with goggles, or any other similar method. In some implementations, the excitation filter 88 can be omitted if the excitation light source 84 is configured to generate light at a narrow range of wavelengths.

The redirected filtered light 88b can then pass through an optics stage 92 which can include a microscope objective 94 and a contact lens 96 or a fundus or slit-lamp camera apparatus. The microscope objective 94 and the contact lens 96 can act to focus, align and magnify the redirected filtered light 88a onto a desired area of the subject retina 30. It will be understood that under some test conditions, an applanation means such as a flat, optically clear lens or plane can be used to flatten or deform the cornea 20 to a desired shape to thereby allow better or more accurate imaging. Alternatively, an appropriate contact lens for fundus viewing can be employed.

The focused redirected light 88c illuminates the retina thereby causing autofluorescence of the associated flavoproteins (FPs) (i.e., flavoprotein autofluorescence (FA)). The FA signal 82a can be directed away from the subject retina 30 and through the components of the optics stage 92, and the dichroic reflector 90 to an emission filter 98 such as, for example, a filter having a passband at approximately 535 nm, such as provided by OMEGA OPTICAL®. The emission filter 98 can be selected to attenuate wavelengths that do not correspond to FA (e.g., wavelengths of or around 535 nm). The filtered FA signal 82b can then pass through a focusing lens 100. The image capture device 81 and/or the optics stage 92 should be configured such that a field of view (FOV) of the image capture device 81 can capture an image of the retina (or any desired portion thereof) in a single picture. As just one specific example, an appropriate FOV can be selected by identifying retinal landmarks such as the optic disc or vascular patterns to use as aiming points and then adjusting the FOV to encompass the entire area of interest. Using an appropriate objective lens or lenses, the FA can be directed onto a camera.

In other implementations, excitation light 88a can be conducted to the eye and/or the FA signal 82b can be conducted to the image capture device 81 via fiber optics. In other words, excitation light 88a can be conducted to the eye and/or the FA signal 82b can be conducted to the image capture device 81 via fiber optics alone or fiber optics in combination with an optical lens system. Of course, excitation light 88a can be conducted to the eye and/or the FA signal 82b can be conducted to the image capture device 81 via an optical lens system without the use of fiber optics.

The camera 82 can be optically coupled to an image intensifier 102 to magnify the brightness of the focused FA 82c to facilitate analysis of the captured image. In some implementations, the image intensifier 102 can be selected such that the gain, which is the ratio between the signal captured by the detector of the CCD camera 82 and the corresponding output signal, represents an increase of approximately 100 to 1000 times the original image intensity. The image can be acquired, for example, by using a high-speed PRINCETON ST-133 interface, a STANFORD RESEARCH SYSTEMS® DG-535 delay gate generator with speeds ranging from 5 nsec to several minutes, and a CCD camera. In this implementation, the delay gate generator cooperates with the CCD camera and the image intensifier 102 to synchronize and control the operation of these components. In other implementations, the shutter of the camera 82 can be opened for a set integration time, typically less than one second (although of course other lengths of times can be used in other implementations).

In other implementations, the camera 82 and the image intensifier can be an integral unit. In such implementations, the image capture device 81 can include, for example, an electron-multiplying charge-coupled device (EMCCD) camera, an intensifying charge-coupled device (ICCD) camera, etc.

It will be understood that the image obtained by the image capture device 81 represents the focused FA signal 82c in an intensified form, the unwanted autofluorescence information or noise having been minimized by the operation of the excitation filter 86 and the emission filter 98. In this manner, the resulting single image captured by image capture device 81 has a high S/N ratio and provides a clear and detailed image representing the FA signal 82a-82c.

The image capture device 81 can be coupled to a computing device 100. The computing device 100 can analyze the image to generate one or more indicators such as indicators of various diseases of the eye or diseases associated with eye damage. Image analysis by the computing device 100 can include one or more of generating a histogram of intensities for units of the image such as pixels or pixel blocks, determining an average intensity, determining an indication of the variance of intensities, determining an integrated intensity, etc. Similarly, multiple images can be analyzed. For example, images of both eyes of a patient can be analyzed, and differences between the eyes can be determined, such as one or more of differences in average intensities, differences in intensity variances, differences in integrated intensities, etc.

The computing device 100 can comprise, for example, an analog circuit, a digital circuit, a mixed analog and digital circuit, a processor with associated memory, a desktop computer, a laptop computer, a tablet PC, a personal digital assistant, a workstation, a server, a mainframe, etc. The computing device 100 can be communicatively coupled to the image capture device 81 via a wired connection (e.g., wires, a cable, a wired local area network (LAN), etc.) or a wireless connection (a BLUETOOTH™ link, a wireless LAN, an IR link, etc.). In some embodiments, the image information generated by the image capture device 81 can be stored on a removable or portable computer readable medium such as a disk (e.g., a floppy disk, a compact disk (CD), a DVD, a portable hard disk drive device, etc.), a FLASH memory device, a memory stick, etc., and then transferred to the computing device 100 via the computer readable medium. Although the image capture device 81 and the computing device 100 are illustrated in FIG. 1 as separate devices, in some embodiments the image capture device 81 and the computing device 100 can be part of a single device. For example, the computing device 100 (e.g., a circuit, a processor and memory, etc.) can be a component of the image capture device 81 or vice versa.

In another implementation, the image capture device 81 can be replaced with a photo detector and a photon intensifier. The photo detector and the photon intensifier can be integrated in a single device, such as a photomultiplier tube. In these implementations, the image capture device 81 can generate a signal that is indicative of an integrated intensity of the FA. The computing device could then use this signal to generate one or more indicators such as indicators of various diseases of the eye or diseases associated with eye damage.

The components of the apparatus 80 described herein can be used in a stand-alone fashion, wherein alignment is accomplished via manual clamping and securing of the individual components. However, the imaging, excitation and optical components of the retinal evaluation apparatus 80 can be integrated into any known desktop or handheld ophthalmoscope, slit-lamp, or fundus camera, to allow easy upgrade to the testing equipment described herein. Specifically, the image capture device 81, the excitation light source 84, the optics stage 92, and the associated components can each be equipped with an adaptor (not shown) designed to allow each of the individual components of the apparatus 80 to be mated with the ophthalmoscopes and other devices discussed above. In this case, the standard ophthalmoscope, fundus, or slit-lamp light can be replaced with the excitation means 84 affixed to the ophthalmoscope frame using a bracket or adaptor and the light output by the excitation means 84 can be filtered to produce the desired excitation light 84a. The image capture device 81 can be attached to the frames of the devices and aligned opposite the retina 30 to detect a single image representing the FA generated in response to the excitation light 84a. In this manner, existing devices can be retrofitted to allow known diagnostic equipment to be used to excite and evaluate retinal autofluorescence.

The apparatus 80 can be aligned and/or calibrated using a variety of techniques. For example, the apparatus 80 can be aligned using techniques such as disclosed in U.S. patent application Ser. No. 10/777,423, filed on Feb. 12, 2004, the contents of which are hereby incorporated by reference herein.

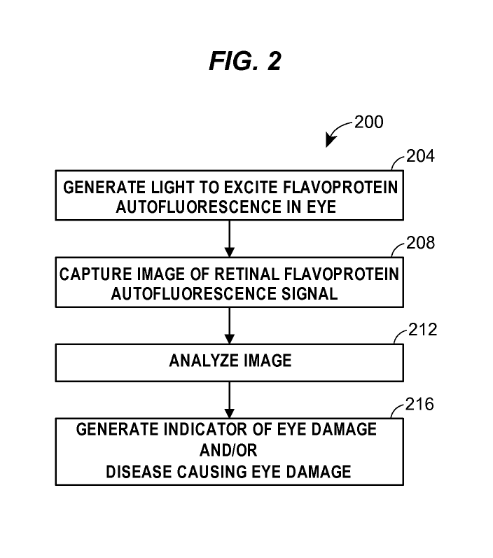

FIG. 2 is a flow diagram of an example method 200 of generating an indicator of one or more of eye damage, a disease of the eye, a disease that causes damage to the eye, etc. The apparatus 80 of FIG. 1 can implement the method 200, for example. Of course, another apparatus can implement the method 200, and the apparatus 80 can implement a method different than the method 200. For ease of explanation, the method 200 will be described with reference to FIG. 1.

At a block 204, light that causes FA is generated and is directed within the eye of a patient. The light can be generated as a flash of light and/or for a relatively short period of time for the comfort of the patient. Typically, the light can be shown for less than one minute, but more generally it can be shown from a few nanoseconds to several minutes. The light that causes FA can be generated by an unfiltered light source that generates narrow wavelength light. Alternatively, a broader wavelength light can be filtered using an excitation filter. In FIG. 1, the light source 84 generates light that is filtered by the excitation filter 88. The filtered light is then directed to the eye of the patient via the mirror 90 and the optics 92.

At a block 208, an image of the retinal FA signal can be captured. The image can be captured in a relatively short period of time, such as about one minute. More generally, the image can be captured after a period of time ranging from a few nanoseconds to several minutes. With the apparatus 80, for example, the retinal FA signal is directed by the optics to the image capture device 81, at which the image is captured.

At a block 212, the captured image can be analyzed. The analysis can include one or more of generating a histogram of intensities for units of the image such as pixels or pixel blocks, determining an average intensity, determining an indication of the variance of intensities, determining an integrated intensity, etc. Similarly, an image can be analyzed with respect to other images, such as an image of the eye taken at a previous time (such a several weeks or months ago, about a year ago, etc.), an image of the other eye of the patient, etc. For instance, images of both eyes of a patient can be analyzed, and differences between the eyes can be determined, such as one or more of differences in average intensities, differences in intensity variances, differences in integrated intensities, etc. In the apparatus 80, the computing device 100 can analyze the captured image.

At a block 216, an indicator of eye damage or of a disease that causes eye damage can be generated based on the image analysis and optionally other information. The indicator can be of eye damage, one of various diseases of the eye, a disease associated with eye damage, etc. The indicator can indicate a degree of damage, a probability of the presence of a particular disease, whether further examination and/or testing is warranted, etc. The indicator can be merely a measure of average intensity, integrated intensity, intensity variance, etc., or a measure of some combination of two or more such factors, such as some combination of average intensity and intensity variance. The indicator can comprise separate sub-indicators such as a sub-indicator of average intensity and a sub-indicator of intensity variance, for example. The indicator can be generated based on other information as well such as one or more image analyses done several weeks, months, or years ago, other test results such as eye or blood tests, patient history and/or family history information, genetic information, etc.

In another implementation, the block 208 can be modified so that an image is not captured, but rather information indicative of the FA signal is determined. For example, a photomultiplier tube could be utilized to determine a total intensity or integrated intensity of the FA signal. In this implementation, the block 212 can be omitted, and the block 216 can be modified so that the indicator of eye damage or of a disease that causes eye damage can be generated based on the FA information determined at the block 208.

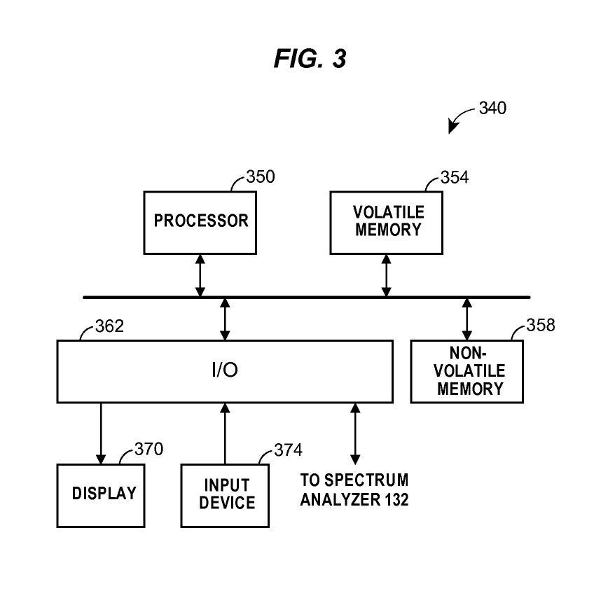

FIG. 3 is a block diagram of an exemplary computing device 340 that can be employed for use in the apparatus 80 and/or the method 200. It is to be understood that the computer 340 illustrated in FIG. 3 is merely one example of a computing device that can be employed. As described above, many other types of computing devices 144 can be used as well. The computer 340 can include at least one processor 350, a volatile memory 354, and a non-volatile memory 358. The volatile memory 354 can include, for example, a random access memory (RAM). The non-volatile memory 358 can include, for example, one or more of a hard disk, a read-only memory (ROM), a CD-ROM, an erasable programmable ROM (EPROM), an electrically erasable programmable ROM (EEPROM), a digital versatile disk (DVD), a flash memory, etc. The computer 340 can also include an I/O device 362. The processor 350, volatile memory 354, non-volatile memory 358, and the I/O device 362 can be interconnected via one or more address/data buses 366. The computer 340 can also include at least one display 370 and at least one user input device 374. The user input device 374 can include, for example, one or more of a keyboard, a keypad, a mouse, a touch screen, etc. In some embodiments, one or more of the volatile memory 354, non-volatile memory 358, and the I/O device 362 can be coupled to the processor 350 via one or more separate address/data buses (not shown) and/or separate interface devices (not shown), coupled directly to the processor 350, etc.

The display 370 and the user input device 374 are coupled with the I/O device 362. The computer 340 can be coupled to the image capture device 81 (FIG. 1) via the I/O device 362. Although the I/O device 362 is illustrated in FIG. 3 as one device, it can comprise several devices. Additionally, in some embodiments, one or more of the display 370, the user input device 374, and the image capture device 81 can be coupled directly to the address/data bus 366 or the processor 350. Additionally, as described previously, in some embodiments the image capture device 81 and the computer 340 can be incorporated into a single device.

At least some of the previously described additional information that can be used for generating an indicator (e.g., previous history of the patient, family history of the patient, genetic information, etc.) can be entered via the user input device 374, loaded from a disk, received via a network (not shown), etc. These additional factors can be stored in one or more of the memories 354 and 358. Additionally, one or more previously measured images can be loaded from a disk, received via a network (not shown), etc., and stored in one or more of the memories 354 and 358.

A routine, for example, for analyzing an image and/or generating an indicator can be stored, for example, in whole or in part, in the non-volatile memory 358 and executed, in whole or in part, by the processor 350. For example, the blocks 212 and/or 216 of FIG. 2 could be implemented in whole or in part via a software program for execution by the processor 350. The program can be embodied in software stored on a tangible medium such as CD-ROM, a floppy disk, a hard drive, a DVD, or a memory associated with the processor 350, but persons of ordinary skill in the art will readily appreciate that the entire program or parts thereof could alternatively be executed by a device other than a processor, and/or embodied in firmware and/or dedicated hardware in a well known manner.

Although the blocks 212 and 216 of FIG. 2 were described above as being implemented by the computer 340, one or more of these blocks can be implemented by other types of devices such as an analog circuit, a digital circuit, a mixed analog and digital circuit, a processor with associated memory, etc.

An apparatus such as the apparatus 80 and/or a method such as the method 200 could be used to monitor disease progression and/or disease treatment. For example, a generated indicator can indicate a degree of damage or distress to ocular tissue, and multiple examinations over time could be conducted to monitor disease progression and/or treatment. For example, the generated indicators could be utilized to monitor whether a disease is worsening, remaining stable, and/or improving, to monitor whether treatment such as medication is relieving distress to the ocular tissue, to monitor whether a treatment such as medication is stabilizing or improving a disease or symptoms of the disease, etc.

Similarly, an apparatus such as the apparatus 80 and/or a method such as the method 200 could be used in animal experiments of disease treatments. For example, the indicators generated based on animals undergoing experimental treatment could be utilized to monitor whether a treatment such as medication is stabilizing or improving a disease or symptoms of the disease, etc.

In general, one or more embodiments of the present invention provide for methods of detecting disease by detecting metabolic dysfunction in a subject's eyes. Metabolic dysfunction is present in the eyes as explained above when a disease is present, whether systemic or ocular. Methods described herein can be performed with the apparatus described above, or some other suitable apparatus, and any of the embodiments can be used where appropriate. Furthermore, each of the methods described herein can be performed on human as well as animal subjects in a clinical or experimental setting.

More specifically, metabolic dysfunction can be detected by detecting modified FA in mitochondria of the subject's eyes. The modified FA can either be increased FA or decreased FA and generally depends on the disease that is present. For example, FA can be decreased in diseases resistant to apoptosis, such as cancer and inflammation.

Increased FA can be determined in a number of ways, such as by detecting asymmetry between the eyes of a subject in FA of an analysis such as average intensity (AI), average curve width (ACW), integrated intensity, and combinations thereof. Differing measurements of each of these analyses between the right eye and the left eye of a subject may indicate disease. Alternatively, these measurements can be made in a subject and compared to a control subject or a database of controls, i.e. normative values from a sample population. Furthermore, photometric readings can be taken with an apparatus such as described herein, or another suitable apparatus, and these readings can be compared between eyes or eyes and controls as above. Measurement of each of these values is demonstrated in the Examples below.

Various diseases can be detected with various embodiments of methods according the present invention. The disease can be a systemic disease that affects not only the eyes but other parts of the body as well, such as diabetes, AIDS, sarcoidosis, systemic lupus erythematosus, rheumatoid arthritis, hypertension, atherosclerosis, sickle cell disease, cancer, inflammation, and multiple sclerosis. The disease can also be a retinal disease such as central serous retinopathy, diabetic retinopathy, retinal vascular occlusion, vitreoretinopathy, retinal vascular disease, infectious and non-infectious uveitis and retinitis, an acquired retinopathy, age-related macular degeneration, inherited retinal degeneration, and retinitis pigmentosa. The disease can also be an optic nerve disease such as pseudotumor cerebri, glaucoma, thyroid eye disease, optic neuritis, and Graves disease. The present invention is not limited, however, to detection of the specific diseases mentioned herein.

In at least some embodiments, the severity of the disease present in the subject can also be determined. For example, the values of AI, ACW, integrated intensity, or combinations thereof of the subject being tested can be compared to AI, ACW, integrated intensity values of a database of controls. Higher values (or lower values, depending on the disease) of AI and ACW in the subject compared to AI and ACW values in the database of controls may indicate a more severe form of disease. This may be helpful for staging a disease and determining the appropriate treatment for a subject.

Of course, various other tests can be performed in addition to measuring FA and/or detecting metabolic and/or mitochondrial dysfunction. For example, levels of HbA1c in the subject may be detected and compared to a database of controls for further diagnosis of the disease. Also, standard clinical tests that are used for determining the presence of disease can be performed along with measuring FA and/or detecting metabolic and/or mitochondrial dysfunction. This can provide the patient with a confidence that a correct diagnosis has been made and confirmed by multiple sources.

In one embodiment of the present invention, a method of early detection of disease includes detecting modified FA in mitochondria of a subject's eyes before clinical symptoms of a disease are able to be detected. This detecting may be performed as described above, for example. In many instances, a subject may have a disease but have no clinical symptoms that can be detected using prior art tests because the disease is at an early stage. The disease may eventually be detected after it has progressed, but in the mean time damage to tissues such as the retina, the optic nerve, etc. also may have occurred and/or it may have become harder to reverse or mitigate effects of the disease. Therefore it would be very advantageous to detect such diseases at an earlier stage. For example, retinal metabolic stress can be detected in a subject before retinopathy is detected. Also, diabetes can be detected in a subject before it can be detected by the common methods of fasting blood glucose screening, plasma glucose testing, or other screening methods. Detecting disease early means there is a greater chance of recovery from the disease or at least selecting appropriate treatment and relieving symptoms before they occur. Especially with early detection of diabetes, patients can alter their diet and exercise routines appropriately in order to mitigate the disease.

In another embodiment, a method of prescreening for disease includes detecting FA in mitochondria of a subject's eyes before detecting clinical symptoms of a disease, and based on results of said act of detecting, recommending and/or performing further clinical testing. Analyzing the FA of the subject's eyes and obtaining a positive result that a subject has a disease can allow the subject to follow up with standard clinical testing to confirm the diagnosis. Using a method according to this embodiment, the presence of disease may be detected earlier than normal and in a non-invasive manner, and the subject can then seek more specialized treatment and testing.

In yet another embodiment, a method of monitoring disease progression includes detecting FA in a subject's eyes at a first time point, detecting FA at at least a second time point, comparing the FA at the first and at least second time point, and determining the progression of disease or generating an indicator indicative of the progression of the disease. Values of FA are taken during at least two time points (a first time point and a second or subsequent time point), but much larger numbers of points can also be used for evaluation (e.g., throughout the subject's life). The time between each detection can be any suitable time, such as a day, a month, a year, multiple years, etc. The values of FA at the second time point can indicate different things depending on the disease. For example, a higher FA at the second time point can be indicative of disease progression and a lower FA at the second time point is indicative of disease mitigation. However, when the disease is cancer or inflammation, a lower FA at the second time point is indicative of disease progression and a higher FA at the second time point is indicative of disease mitigation. Also, treatment can be provided to the subject based on the FA at the at least second time point such as lifestyle changes (exercise), pharmaceuticals, nutraceuticals, surgery, chemotherapy, radiotherapy, laser treatment, etc. The detection and comparison can be performed for multiple time points and the treatment provided at the later time point can be assessed. If the treatment is not providing results, it can be altered, or if the treatment is providing results, it can be continued. Standard clinical tests can also be performed, and disease progression may be monitored additionally based on the results of these standard clinical tests.

In still another embodiment, a method of detecting ocular changes due to an effect of a substance includes detecting modified FA in mitochondria of a subject's eyes while a substance is known to be present in the subject. The substance present can be a pharmaceutical or a toxin, such as described in the Examples.

In another embodiment, a method of testing treatments on a subject for effectiveness includes administering at least one treatment to a subject with a disease, detecting FA in a subject's eyes at a first time point, detecting FA at at least a second time point, comparing the FA at the first and second time point, and determining the effectiveness of the treatment on disease or generating an indicator of the effectiveness of the treatment. The treatment can be any suitable treatment such as, but not limited to, lifestyle changes (exercise), pharmaceuticals, nutraceuticals, surgery, chemotherapy, radiotherapy, laser treatment, etc. As above, values of FA are taken during at least two time points (a first time point and a second or subsequent time point), but an unlimited number of points can be used for evaluation (i.e. throughout the subject's entire life). The time between each detection can be any suitable time, such as a day, a month, a year, multiple years, etc. This method may be used to determine the effectiveness of treatments that are unknown to be effective on the particular disease being studied, i.e. it may be utilized as a drug discovery method. However, treatments known to be effective can also be tested. Combinations of these treatments can be tested. Depending on the disease, the results of the FA at the second time point can be different. For example, a higher FA at the second time point can be indicative of ineffectiveness and a lower FA at the second time point can be indicative of effectiveness. When the disease is cancer or inflammation, a lower FA at the second time point can be indicative of ineffectiveness and a higher FA at the second time point can be indicative of effectiveness.

This method can also be utilized as a method of personalized medicine. In other words, the treatment or combination of treatments can be determined for a particular subject according to the acts above and the treatments described above. The best treatment for each subject can be tailored to their body. Once the particular treatment is determined that will provide the best treatment for the subject, an administration plan can be made.

This method can also be used to optimize the selection of a single treatment or combination of treatments for ocular disease in humans and animals. The treatment may include pharmaceuticals, nutraceuticals, lifestyle changes, chemotherapy, radiotherapy, surgery, laser treatment, etc., and combinations thereof. In other words, the treatment regime can be analyzed at different time points in order to optimize the treatment that the subject receives. The treatment can further be optimized based on other diagnostic modalities or data thereof, including those obtained by serum tests, genetic tests, biochemical tests, optical tests, including optical coherence tomography, psychovisual tests, including visual field testing, ultrasonic tests, other types of spectral analysis, tonometry, keratometric tests, fundus photography, other types of ocular imaging, electroretinographic tests, magnetic resonance imaging tests, isotopic or dye imaging tests, other diagnostic methods, any other physical measures, and any combinations thereof. The treatment can also be optimized in combination with patient data, patient medical history, patient family history, lifestyle history, patient demographics, and any other such measures or combinations thereof.

There are several potential advantages that may be provided by one or more embodiments of the present invention. FA imaging in vivo is sensitive, but not specific to a disease entity. It does, however, permit detection of metabolic dysfunction before any current clinical method can. The recognition of cell or mitochondrial stress before the onset of apoptosis is very beneficial in recognizing a disease state before irreversible damage has occurred. Automated visual field testing, multifocal ERG and new MRI imaging technologies can also detect functional abnormalities in eyes that do not yet display morphologic abnormalities. However, these methods are time intensive and require trained technicians. As the tests take time to perform, they may not be suitable for children or adults unable to cooperate and maintain fixation for the duration of the study. In some embodiments, FA measurement requires less than five minutes to perform, obtaining the data as four rapid snapshots of each eye. The readings of the instrument, along with clinical data obtained by the eye care professional, optionally, are used to arrive at a diagnosis or monitor the severity of the disease.

Experimental examples are provided below for the purpose of illustration only, and are not intended to be limiting unless otherwise specified. Thus, the present invention should in no way be construed as being limited by the following examples, but rather, be construed to encompass any and all variations which become evident as a result of the teaching provided herein.

### Example 1

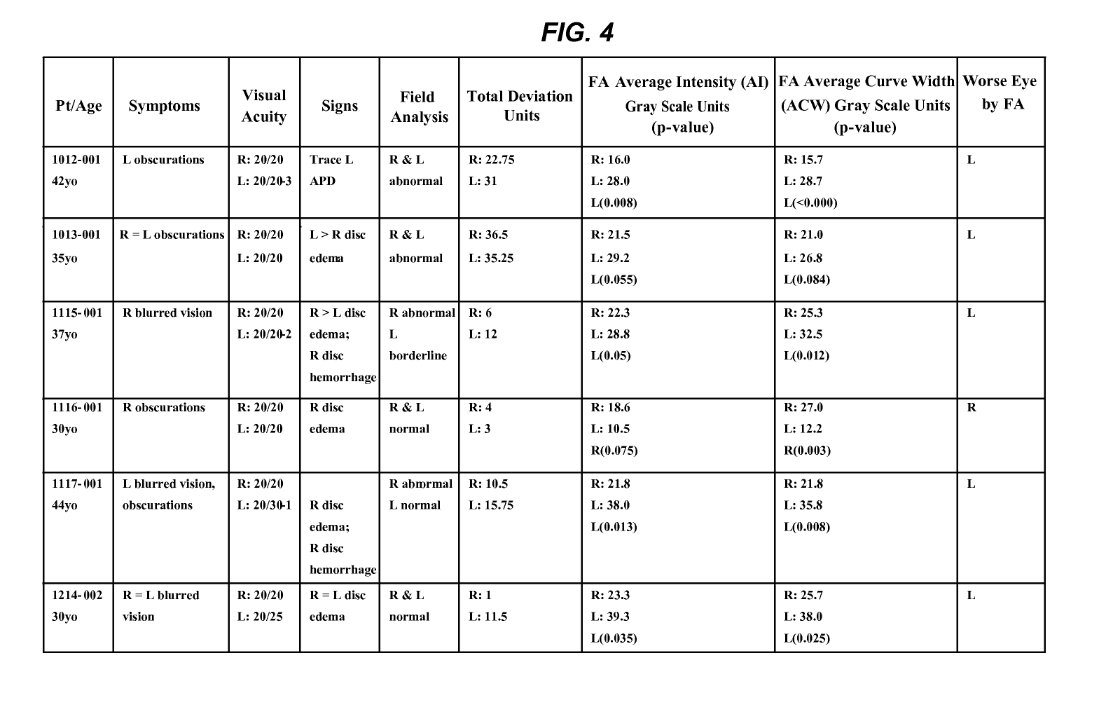

Metabolic stress preceding apoptosis can be monitored in living cells by measuring mitochondrial FA. To help verify that FA can detect eye disease, pseudotumor cerebri (PTC) in which retinal ganglion cell dysfunction and death can lead to visual loss was tested. Six women newly-diagnosed and untreated for PTC (36.3+5.9 years) with 20/30 or better vision in each eye and subtle or no defects on Humphrey automated visual field testing (AVFT) analysis (FIG. 4) were studied. AVFT results were recorded as was the sum of all total deviation units for each eye by assigning a value of 1 for dense loss with lesser degrees of loss graded as 0.75, 0.5, and 0.25, the latter for the most subtle sensitivity reductions. Six age-matched (36.5+4.7 years) healthy women served as controls.

After pupillary dilation, three 3 degree images centered on the fovea, were obtained from each eye using a Zeiss F4 camera modified by inserting 467DF8 nm excitation and 535 nm emission filters (Omega Optical, Brattleboro, Vt.), attaching a back-illuminated electron-multiplying charge coupled device camera (Photometrics, Tucson, Ariz.), and interfacing a computer equipped with MetaView (MDS Analytical Technologies, Toronto, ON) image capture and Lispix (National Institutes Standards & Technology) analysis software. Total test time for each patient was less than six minutes.

For each image, histograms depicting pixel FA intensities (256 unit gray scale) were analyzed to determine average intensity (AI) and average curve width (ACW) in gray scale units (gsu), the latter designating the range of pixel FA intensities. After separately obtaining FA data and clinical data in a masked fashion for each patient, the AI and ACW of the eyes were compared by calculating ratios of the more clinically affected eye to the less affected “control” eye designated by a combination of symptoms, signs, and subtle AVFT total depression values. Statistical analysis was performed using Students' two-tailed t-test and ANOVA.

A 42 year-old PTC patient (FIG. 4, Row 1) had visual acuities of 20/20 and subtle clinical findings of visual dysfunction. AVFT is abnormal, but does not show significant distinction between the eyes. On FA testing, however, the left (red) AI is 1.75 times that of the right (blue) AI (p<0.001). The left ACW is also 1.82 times that of the right ACW (p<0.001). These FA differences highlight disease asymmetry better than visual acuity, pupillary reflex, or AVFT analysis.

All PTC patients (FIG. 4) had higher AI (1.60+0.21 times control) and ACW (1.62+0.36 times control) in their more clinically affected eye. For each patient, AI and ACW of the more clinically affected eye were at least 1.25 times that of the less affected eye. The FA results correlated with symptoms and signs, as well as or better than AVFT in each case. The six age-matched control women without disease showed no significant differences for AI or ACW. Lack of AI (1.01+0.12 times control) or ACW (1.02+0.11 times control) differences were significant (p<0.01) compared to the PTC patients.

In PTC, ophthalmoscopic appearances are not diagnostic for visual dysfunction and its detection relies on subjective tests, particularly AVFT which is regarded as the most sensitive. Nevertheless, early dysfunction can remain undetected by conventional AVFT as demonstrated by short-wavelength and motion techniques.

In the eye, mitochondrial FA constitutes a shoulder of a broad emission spectrum of other fluorophores, especially lipofuscin. To reduce confounding autofluorescence, young patients were chosen, eyes in the same patient were compared, and a narrow emission band at the FA maximum was captured, excluding most of the lipofuscin emission. Although some fluorescence noise remains, this approach distinguished disease severity between 20/20 eyes and between eyes that conventional AVFT could not. FA characteristics indicating dysfunction include: 1) high AI from impaired metabolic activity; 2) broad ACW from disease affecting individual cells to different degrees; and 3) AI and ACW asymmetry between eyes of the same individual. Results suggest that FA imaging can become an important diagnostic tool for ocular disease.

### Example 2

In this study, retinal FA levels in subjects with diabetes, regardless of disease severity or duration, were compared to that of age-matched healthy controls.

### Materials and Methods

To measure retinal FA, a modified fundus camera containing 467 nm excitation and 535 nm emission filters (Omega Optical, Brattleboro, Vt.), two Photometrics 512B back-illuminated electron-multiplying charge-coupled device (EMCCD) cameras (Roper Scientific, Tucson, Ariz.), and computer hardware and software were used such as described above.

Twenty-one subjects, aged 30-59 years, with established type I or type II diabetes and without ophthalmic disease besides retinopathy were enrolled during routine funduscopic examinations. Plasma glucose (obtained at the examination) was assessed by the glucose oxidase method and the glycohemoglobin (HbA1C) levels were measured by high-performance liquid chromatography. Twenty-one age-matched healthy subjects with normal glucose tolerance, normal blood pressure and normal lipid profile were recruited as the control population.

After pupillary dilation, one EMCCD camera was used to visualize the macula using RSImage (Roper Scientific, Tucson, Ariz.) software. For each eye, the second EMCCD camera, interfaced with MetaVue (MDS Analytical Technologies, Toronto, ON) software, was used to capture three to five 535 nm FA readings, each induced by a one millisecond, 467 nanometer incident flash. Imaging required five minutes per patient.

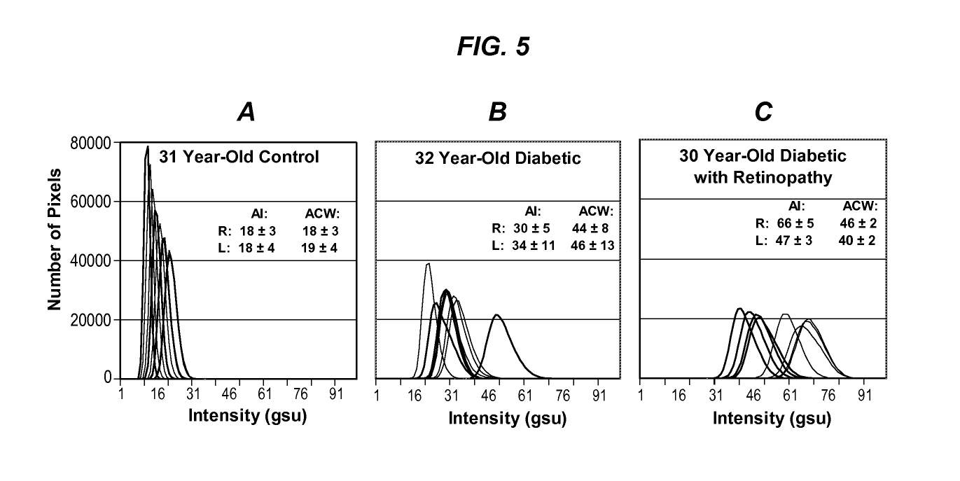

FA images, stored as 512×512 pixel files, were analyzed to produce histograms using MetaVue, Adobe Photoshop CS2 (Adobe Systems, San Jose, Calif.), and Lispix (National Institute of Standards and Technology, Gaithersburg, Md.). The histograms of pixel intensities (FIG. 5), ranging from 0 to 256 grayscale units (gsu), were plotted for each eye to yield average intensity (AI) and average curve width (ACW) of retinal FA. Two research associates trained in FA image evaluation independently interpreted all images. If disagreement was encountered, a consensus reading was performed. At the time of imaging and statistical analysis, the research associates knew if the patient had diabetes, but test review bias was minimized by: 1) not excluding any subjects' data and 2) relying on objective results of FA testing. Student's t-test and ANOVA were used to compare AI and ACW of the diabetics and controls. Comparisons of eye-specific AI and ACW levels between diabetics and controls were made using mixed linear regression to adjust for intereye dependency and age (where appropriate). SAS 9.0 software (SAS Institute Inc., Cary, N.C.) was used for all statistical analyses. A p-value <0.05 was considered significant.

### Results

Twenty-one diabetics out of 33 consecutive diabetic subjects referred for imaging met the inclusion criteria and were imaged to determine AI and ACW for each eye. The mean age for diabetics was 44.8±10.0 years (range 30-59 years), the mean documented diabetes duration was 10.5±9.8 years, and the mean HbA1C was 8.5±1.9%. There were six type I and fifteen type II diabetics. Diabetic retinopathy was present in twelve subjects. All 21 consecutively recruited control volunteers met inclusion criteria. The mean age for controls was 44.7±9.4 years (range 30-59 years).

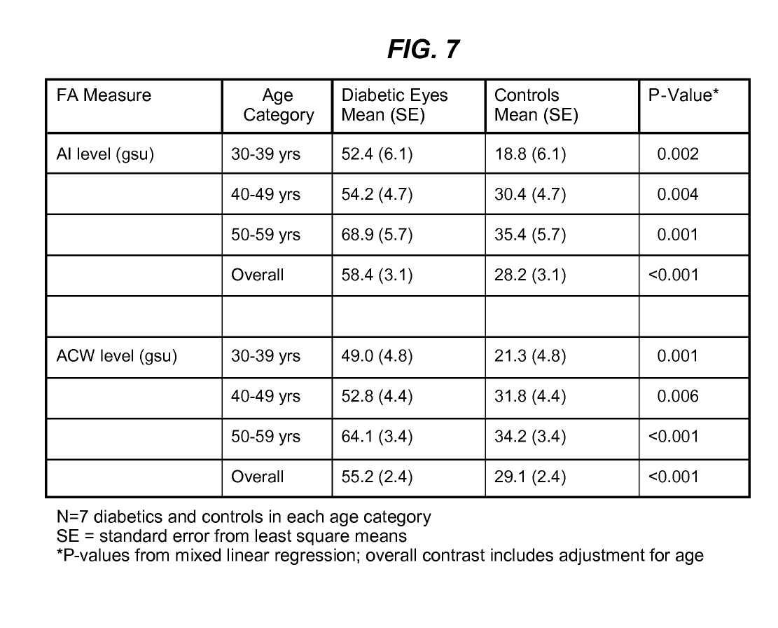

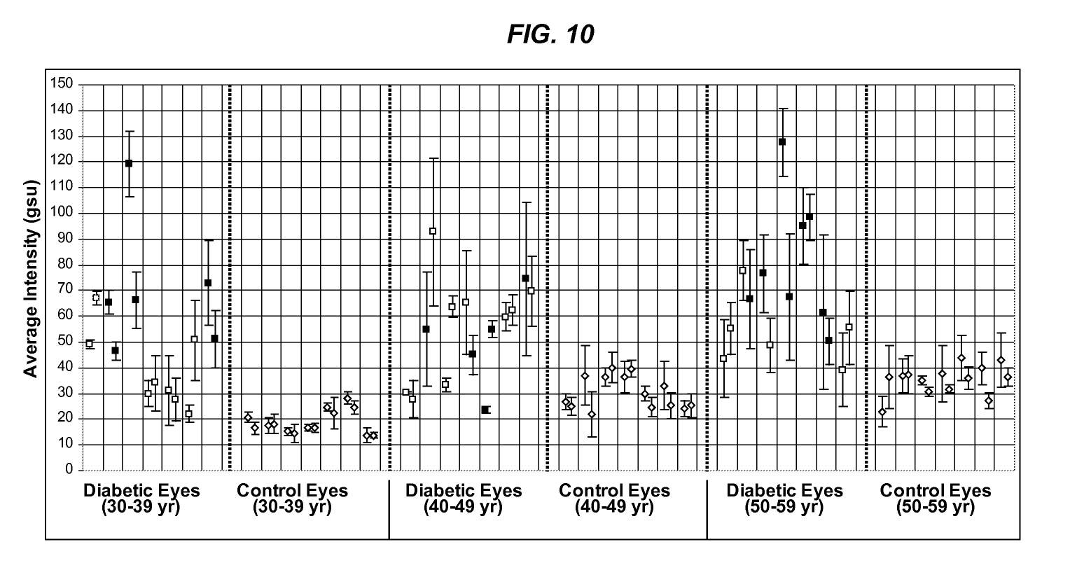

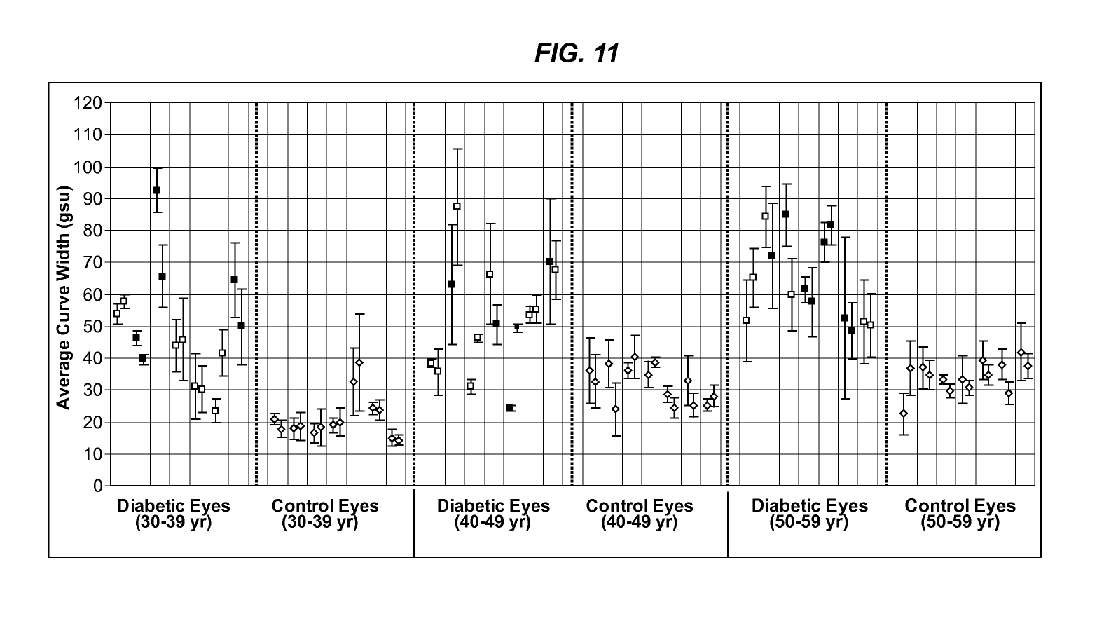

As shown in FIG. 7 and FIG. 10, for all three age strata (30-39, 40-49, & 50-59 years), the mean AI level of diabetics was significantly greater than that found in controls (P<0.004). An overall comparison of the mean AI level in all diabetics vs. all controls, with adjustment for age, was consistent with the results found in each age category (P<0.0001). Similar findings are seen for mean ACW levels (see FIG. 7 and FIG. 11), which were significantly higher for comparisons of diabetics to controls within each age strata (P<0.006) and in an overall comparison with adjustment for age (P<0.0001).

AI and ACW of diabetics and age-matched controls were compared to determine age dependence of FA as other endogenous autofluorescent molecules, such as lipofuscin, accumulate with age and can affect FA intensity. In each age group, AI and ACW of diabetics were greater than those of controls, the latter showing gradual, steady increases of FA with age. However, the relative elevations of FA in diabetics compared to controls appeared to be independent of patient age, strongly suggesting that elevated FA in diabetes is not due to lipofuscin or other similar fluorophores.

Differences in FA values between type I and II diabetics were considered, but FA of the fifteen type II diabetics (AI 59.5±23.6 gsu) and six type I diabetics (AI 55.8±25.9 gsu) did not differ (p=0.654).

Elevated AI and ACW were detected in diabetics regardless of whether any retinopathy was detected on fundus examination by an ophthalmologist specializing in diabetic retinopathy. In fact, nine of the 21 diabetics had no visible retinopathy (FIG. 10), indicating that retinal metabolic stress due to diabetes is present before any visible retinopathy.

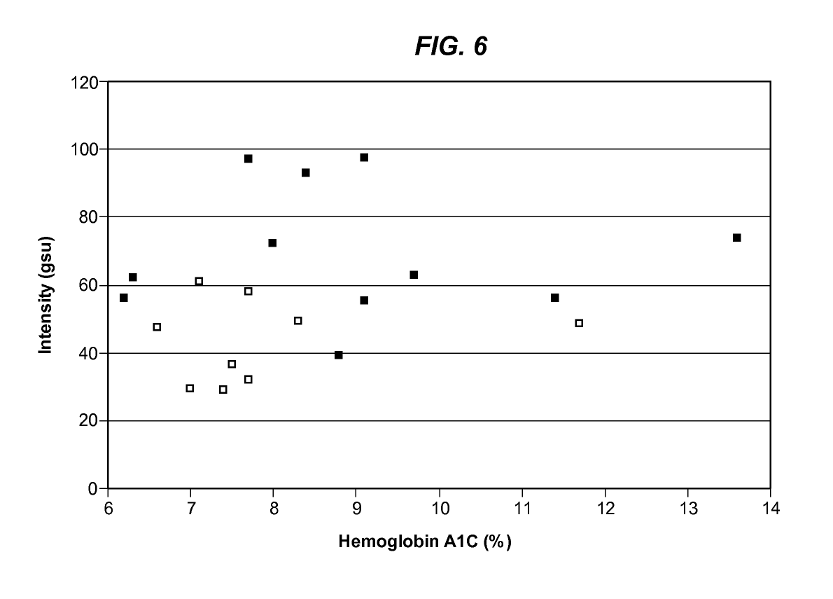

The associations between HbA1C, FA, and the presence of retinopathy (FIG. 6) were studied. The average HbA1C for diabetics with retinopathy in at least one eye (8.9±2.1%) was not significantly different (p=0.232) from those without retinopathy in either eye (7.9±1.5%). However, the mean AI and ACW for diabetics with retinopathy in at least one eye (69.7±18.3 and 63.2±14.5 gsu, respectively) were significantly different (p=0.002 and p=0.005, respectively) from those without retinopathy in either eye (43.5±12.2 and 44.8±10.5 gsu, respectively).

To consider the possibility that FA imaging might measure acute fluctuations in plasma glucose rather than the metabolic effects of chronic hyperglycemia, FA of four volunteers was measured in a fasting state and one hour after 75 g oral glucose challenge. No significant differences were observed in FA values, indicating that acute elevations in plasma glucose do not influence retinal FA.

FA imaging of diabetics results in outcomes that differ significantly from age-matched controls. Only one diabetic (FIG. 10, first from left, 40-49 age group), an intensively-treated type I diabetic within 1 year of diagnosis and with a HbA1C of 7%, had AI and ACW values of each eye that overlapped with those of age-matched controls. Thus, the example device was able to measure significant differences between groups of controls and diabetics, regardless of disease duration or severity. In several subjects, both control and diabetic (FIGS. 10 and 11) there is a statistical difference in AI and ACW values between their two eyes, suggesting that FA is detecting increased retinal stress in one eye as opposed to the other. In fact, it has been found that a high degree of asymmetry between eyes of the same individual is a strong indicator of disease. Improvements in FA technology, including light sources with low flash-to-flash variability and feedback correction for variability, can greatly reduce AI and ACW standard deviations to make FA imaging a sufficiently sensitive screening tool for diabetes. In this scenario, due to the high prevalence of diabetes, individuals with abnormally high FA might undergo glucose tolerance testing that, if negative, would prompt investigation for other causes of ocular tissue dysfunction.

For diabetics, FA levels were associated with the severity of retinal damage. Thus, FA can be useful in monitoring disease progression and its mitigation by treatment. In fact, FA measures in diabetics were more strongly associated with retinopathy than HbA1C levels, which are currently considered the most reliable measures of metabolic control. The value of FA imaging is supported by two patients with retinopathy (FIG. 6) who had elevated FA, but low HbA1C levels. Thus, this study shows that FA is useful in monitoring disease progression and its mitigation by treatment. Unlike glucose monitoring, elevations in FA reflect ongoing diabetic tissue damage and, therefore, can provide patient and caregiver motivation for intensifying disease management.

Ocular emission spectrophotometry has shown that mitochondrial FA constitutes a shoulder of a broad emission spectrum of other fluorescent species, especially lipofuscin. For maximal metabolic contrast, only a narrow emission band at the FA maximum is acquired, effectively excluding most of the emission of lipofuscin. To account for the residual portion of the FA signal derived from age-dependent accumulation of lipofuscin, age-matched FA comparisons were used to correct for this variable.

As neuronal loss and microangiopathy occur early in human and animal diabetes, tissue damage begins at the earliest stages of the disease, before it is clinically evident or detected by fasting blood glucose screening. Early diagnosis and treatment is likely to prevent this damage. The data herein shows that development of FA imaging for diabetes, including its evaluation in longitudinal clinical trials, results in a tool that is increasingly important in disease detection and management.

### Example 3

Other experiments have shown that an apparatus and/or method such as described above is effective in diagnosing a variety of retinal and optic nerve diseases such as listed in Table 1.

TABLE 1

| Retinal | Optic Nerve |
|---|---|
| Central serous retinopathy | Pseudotumor cerebri |
| Diabetic retinopathy | Glaucoma |
| Retinal vascular occlusion | Thyroid eye disease |
| Vitreoretinopathy | Optic neuritis (multiple sclerosis) |
| Retinal vascular disease | Graves disease |
| Infectious and non-infectious | |
| uveitis and retinitis | |
| Any other acquired retinopathy | |
| Age-related macular degeneration | |
| Inherited retinal degeneration | |

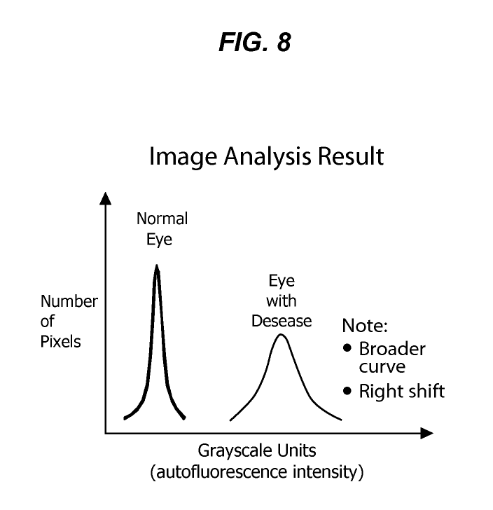

A prototype device was tested on many patients with retinal and optic nerve diseases. Data collected by the prototype indicate that there are at least three independent parameters that can be measured and analyzed to distinguish patients with early disease. As depicted in FIG. 8, a diseased eye exhibits: 1) increased flavoprotein autofluorescence (FA) intensity, 2) a broader range of FA intensity (curve width), and 3) asymmetry of intensity and/or range of intensity between eyes.

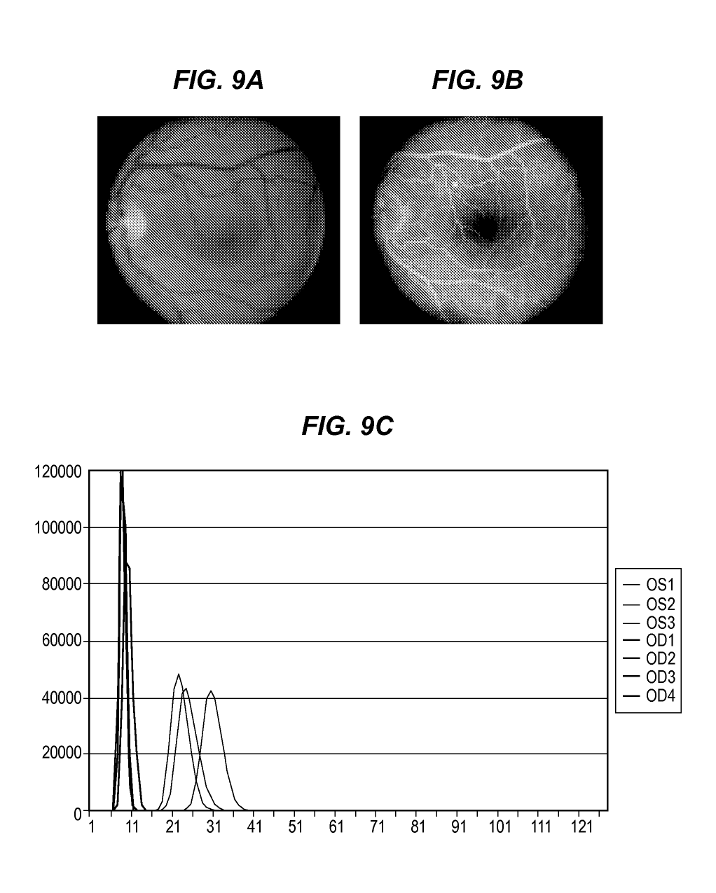

The prototype device was first tested on patients with a retinal disease, central serous retinopathy (CSR). As seen in FIG. 9, FA intensity was greater in the eyes affected by CSR. The FA curves obtained from the affected eyes were also broader, indicating that the disease process affected individual cells to different degrees. In contrast, the unaffected eyes demonstrated narrower curves of lower intensity. Thus, two variables distinguished the affected from the unaffected eyes: 1) mean FA intensity, and 2) mean FA curve width. In addition, asymmetry in either of these two variables is also characteristic of disease. This phenomenon was reproducible in multiple RMITM repetitions performed on each eye as shown in the graphs of the lower panel. Moreover, the repetitions provided statistical data permitting comparisons that showed p-values <0.001 for each of the two variables. RMITM was used to assess metabolic stress in other patients with retinal diseases (TABLE 1) affecting one eye. The differences in the two variables permit an algorithm that distinguishes disease from non-disease for all eyes that have been imaged to date.

The prototype device was also used to study optic nerve disease in order to demonstrate the technology's applicability to this important group of common ocular diseases (TABLE 1). In a study on pseudotumor cerebri (PTC) that resulted in a manuscript for publication in a medical journal, FA values were found to correlate with symptoms and signs, as well as or better than automated visual field analysis in each case. In all cases, comparisons of mean FA intensity and mean FA curve width permitted distinguishing of the more affected from the less affected eye, even though this was not possible by automated visual field analysis in three of the six patients, all of whom had excellent visual acuities and only subtle, early disease findings.

The prototype device was also used to show effects of treatment on retinal and optic nerve disease. In qualitative studies, the eyes of 3 patients with clinically significant diabetic retinopathy had high FA intensities, which correlated with symptoms and signs. Clinical improvement following triamcinolone acetonide injection was accompanied by reduced FA intensities in all 3 cases. In 4 patients, the effects of surgical decompression on optic neuropathy due to thyroid-associated ophthalmopathy were assessed. In all cases, the affected eyes exhibited impaired visual acuity, color perception, and automated visual fields as well as high FA intensities. In all 4 cases, orbital decompression reduced FA within 3 days of surgery, and within 6 hours in one patient tolerating RMITM within hours of decompression. The reductions in FA intensity correlated closely with improvement of other clinical parameters, however, FA intensity often was reduced before the other parameters showed improvement. Eyes treated for diabetic macular edema and compressive optic neuropathy were imaged sequentially, and showed dramatic reductions in FA intensities within hours to days of treatment. As these reductions were seen in the same eyes within short time intervals after treatment, this strongly suggests that dynamic FA changes were not due to other endogenous autofluorescent molecules, such as lipofuscin, which only slowly accumulate or are stably present in the eye.

Results to date strongly indicate the diagnostic and prognostic promise of the RMITM technology compared to other clinical methods.

### Example 4

In this study, FA was examined in older patients with diabetes mellitus and other patients with retinal specific diseases to determine the clinical sensitivity of this technique in detecting disease-associated retinal metabolic stress. To show that pre-apoptotic conditions induce FA elevations in retinal cells, in vitro FA measurements were compared. Cultures of normal human retinal pigment epithelial (HRPE) cells, HRPE cells exposed briefly to ceramide or H2O2, agents known to induce apoptosis in HRPE and HRPE cells exposed to ceramide or H2O2 in the presence of either anti-oxidants or inhibitors of these agents were measured and compared.

### Methods

### Human Subjects.

Thirty six patients total ranging in age from 23-77 years were imaged for FA. Fourteen patients (ages 54-68) with a history of diabetes mellitus, along with seven age matched control volunteers (age 57-67) were imaged to examine the effects of diabetes and diabetic retinopathy on FA. One patient with age-related macular degeneration (ARMD), one patient with central serous retinopathy (CSR) and one patient with retinitis pigmentosa along with an age-matched control for each were also imaged. Nine additional healthy volunteers without a history of diabetes mellitus or ocular disease were imaged and used to examine the effects of age on retinal FA.

### Retinal FA Imaging in Humans.

To measure FA in humans, a Zeiss FF4 fundus camera (Carl Zeiss Corporation, Oberkochen, Germany) underwent more than 50 modifications as previously described. These included inserting special 467 nm excitation and 535 nm emission filters, attaching a back-illuminated EMCCD camera, and connecting to computers with custom software. The EMCCD chip was cooled to −30° C. to reduce noise. Additional modifications included optimizing light transmission to improve signal and placing optical baffles to reduce noise due to light reflections.

Each patient's/volunteer's pupils were dilated with 1% topicamide/2.5% phenylephrine and the EMCCD camera was used to capture four 535 nm FA readings, each induced by a one millisecond, 467 nm incident flash. Imaging requires five minutes per individual. The depth of focus of the instrument results in capture of FA from all retinal layers. The images were stored as 512×512 pixel 16-bit grayscale TIFF files.

### Retinal FA Image File Analysis.

Histogram curves of pixel intensities in grey scale units (gsu) captured by each well of a 512×512 CCD chip (262,144 pixels) were extracted from the TIFF files using Metavue, Adobe Photoshop CS2 (Adobe Systems, San Jose, Calif.), and Lispix (National Institute of Standards and Technology, Gaithersburg, Md.). The histograms of pixel intensities, ranging from 0-256 grey scale units (gsu), were plotted for each eye to yield average intensity (AI) and average curve width (ACW) of retinal FA.

### Materials.

N-acetyl-cysteine, hydrogen peroxide, C2-ceramide and dihydroceramide C2 were purchased from Sigma-Aldrich (St. Louis, Mo.). Costar tissue culture 96-well assay plates (black walls with clear bottoms) were purchased from Fisher Scientific (Pittsburgh, Pa.).

### HPRE Cell Culture.

HRPE cells were isolated from donor eyes by enzymatic digestion as previously described. Briefly, the sensory retina was separated gently from the HRPE monolayer, and the HRPE cells were removed from Bruch's membrane using one hour incubation with papain. Isolated HRPE cells were grown into Falcon Primaria flasks in DMEM/F12 containing 10% fetal bovine serum (FBS), penicillin G (100 U ml-1), streptomycin sulfate (100 μg ml-1), and amphotericin B (0.25 μg ml-1) at 37° C. in a humidified incubator under 5% CO2. In all experiments, parallel assays were performed on the second to fourth passage of HRPE cells. HRPE cells were seeded into tissue culture 96-well assay plates (black walls with clear bottoms (Costar) at the same time and density from the same parent cultures, and grown in phenol red-free complete DMEM/F12 for at least 7 days. All experiments were repeated at least three times on different cell lines.

### HRPE Cell Incubations.

HRPE cells in 96-well plates were washed with Hanks' balanced salt solution (HBSS), containing Ca2+ and Mg2+, without phenol red. HRPE cells were pre-incubated for 30 minutes with or without N-acetyl-cysteine (NAC; 1 mM), followed by stimulation with hydrogen peroxide (H2O2; 0.2 mM) for 3 hours in the presence and absence of the same inhibitor used during pre-incubation. Other HRPE cells in 96-well plates were pre-incubated for 30 minutes with or without dihydroceramide C2 (50 μM), followed by stimulation with C2-ceramide (50 μM) for 2 hours in the presence and absence of the same inhibitor used during pre-incubation.

### HRPE Cell FA Measurement.

FA was measured with a photomicroscope equipped with narrow bandwidth excitation and emission filters of 465 nm and 535 nm, respectively.

### Results

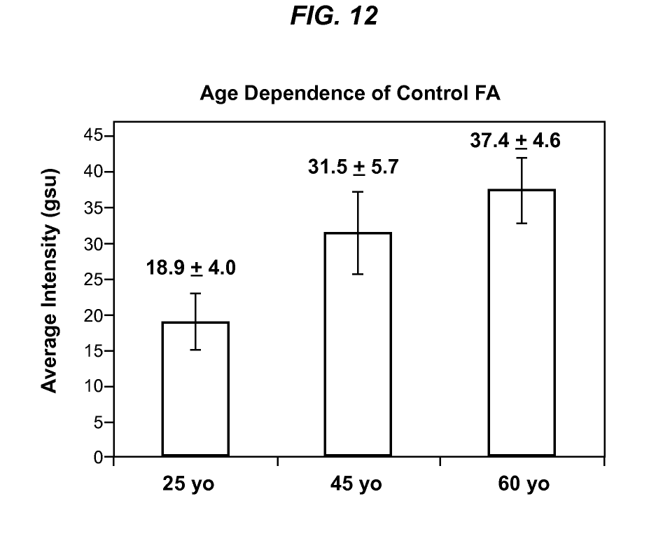

Nine control volunteers without evidence of diabetes mellitus or ocular disease were imaged for retinal FA to examine the potential changes in retinal FA that may occur with age. Three control volunteers in each of three age groupings were studied and the average retinal FA pixel intensity for each is presented in FIG. 12. Average age groupings of 25, 45, and 60 years were studied with each volunteer within an age group being within 1-2 years of the average for that group. Control volunteers in the 25 year group showed substantially less FA (average pixel intensity (AI) 18.9+4 grey scale units (gsu)) than either the 45 year old group (AI=31.5+5.7 gsu, p=0.04) or the 60 year old group (AI=37.4+4.6 gsu, p=0.01). Although the 45 year old group showed less retinal FA than the 60 year old group, this was not significant (p=0.24). The average curve width (ACW) also increased progressively with increasing age, with 25 year old having narrower curves (ACW=25.3+3.1 gsu) than either 45 year old (ACW=31.0+5.3 gsu) or 60 year old (ACW=36.7+5.7 gsu; p=0.05) groups.

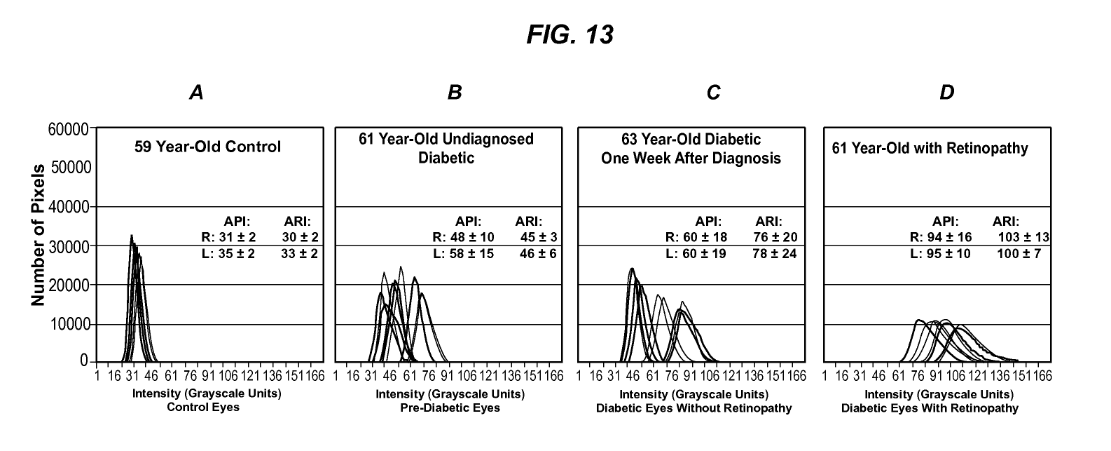

Representative histograms of numbers of pixel counts at each of 256 pixel intensities for retinal FA for diabetic patients without retinopathy, with retinopathy and an age-matched control volunteers are shown in FIGS. 13A-13D. Four histograms, corresponding to the 4 images taken in each eye, are graphed, for each patient.

FIG. 13A shows a 59 year old control volunteer without ocular disease or history of diabetes. FIG. 13B shows a 61 year old patient without ocular disease or known history of diabetes mellitus-increased retinal FA, however, prompted serum glucose testing, which was found to be abnormal. FIG. 13C shows a 63 year old patient with diabetes mellitus without retinopathy. FIG. 13D shows a 61 year old patient with bilateral non-proliferative diabetic retinopathy.

Curves shifted to the right indicate greater intensity of FA. Broader curves indicate a greater variation in FA intensity in retinal cells within the area of the retina imaged. Since the retinal imaging only subtends 3 degrees on the retina, non-overlapping histograms seen for a single eye, may result from small changes in the patient's/volunteers fixation during imaging, resulting in slightly different areas of the macula being imaged.

The retinal FA histograms for a 59 year old non-diabetic control volunteer demonstrated lower average intensities (AI) and narrower curves (ACW), compared to any of the patients with diabetes mellitus. (FIG. 13) Retinal FA histograms for the 61 year old diabetic patient, with retinopathy, demonstrated broader curves with higher average pixel intensities compared to the two representative diabetic patients without retinopathy. (FIG. 13)

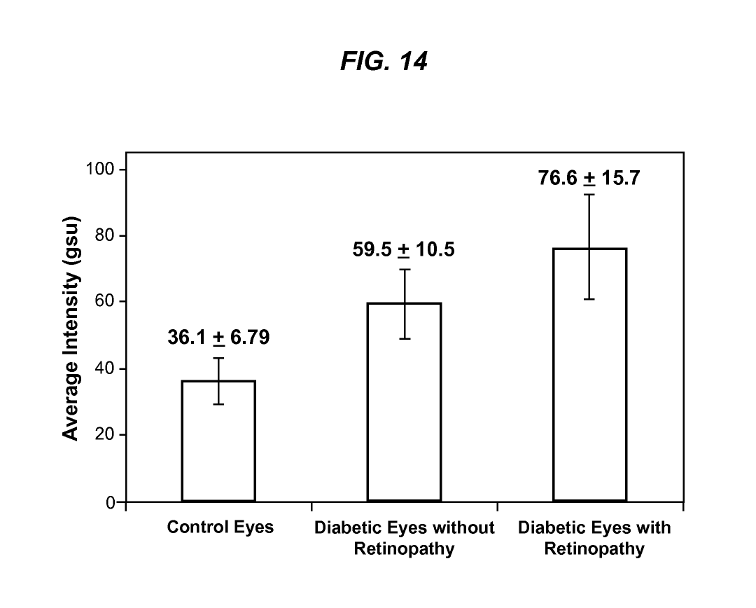

Fourteen total patients, aged 54-68 years old, with diabetes mellitus were imaged for retinal FA. All but one of the patients had Type 2 diabetes mellitus. Seven of the 14 patients had evidence of diabetic retinopathy on exam, with the retinal disease bilateral in all but one patient. Patients with retinopathy had an average HgbA1c level=7.7+1.7%, patients without retinopathy had an average HgbA1c level=6.8+0.8%. All diabetic eyes had visual acuity 20/40 or better, except one eye which was 20/200 in a patient without retinopathy. Seven healthy age-matched control patients, age 57-67 years, were also imaged for retinal FA and compared to the diabetics. Patients with diabetes, but without retinopathy, had significantly greater average FA intensity (AI=59.5+10.5 gsu) compared to the average retinal FA intensity in age-matched control volunteers (AI=36.1+6.79 gsu); (p=0.001). (FIG. 14) Diabetic patients with retinopathy showed increased average retinal FA intensities (AI=76.6+15.7 gsu) compared to eyes of diabetics without retinopathy (p=0.04). (FIG. 14) ACW was increased in diabetics without retinopathy (ACW: 58.7+9.1 gsu), compared to controls (ACW: 35.7+6.8 gsu; p=0.001). Diabetics with retinopathy had broader curves than diabetics without retinopathy (ACW: 72.1+15.9 gsu; p=0.08). These findings suggest retinal FA not only is a reliable indicator of diabetes mellitus, but also an indicator of the severity of disease.

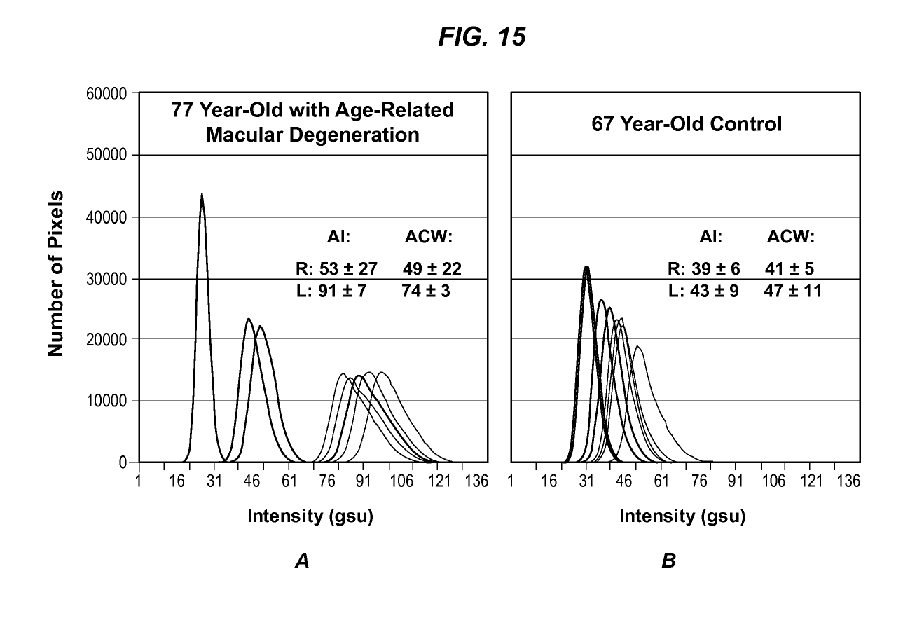

Other retinal diseases were screened for retinal FA and compared to age-matched control patients. A 77 year-old patient with non-exudative ARMD, extensive central geographic atrophy and visual acuity 20/400 OS, and focal exudative ARMD with visual acuity 20/50 OD, recently treated with bevacizumab injections, was imaged for retinal FA. This ARMD patient (FIG. 15A) was compared to retinal FA of the oldest control volunteer, age 67 (FIG. 15B). Four retinal histograms from each eye in each individual are presented. Both eyes in the patient with ARMD had greater AI (OD=53+27 gsu, OS=91+7 gsu) than the control volunteer (OD=39+6 gsu, OS=43+9 gsu) The left eye of the ARMD patient, which had more clinically diffuse non-exudative disease showed greater AI and ACW than the right eye (ACW OD 49+22 gsu, OS 74+3 gsu) which had more focal disease clinically. The variability in the AI and ACW in the right eye of the ARMD patient may represent slight changes in the patient's fixation during imaging resulting in slightly different areas of the macula being imaged.

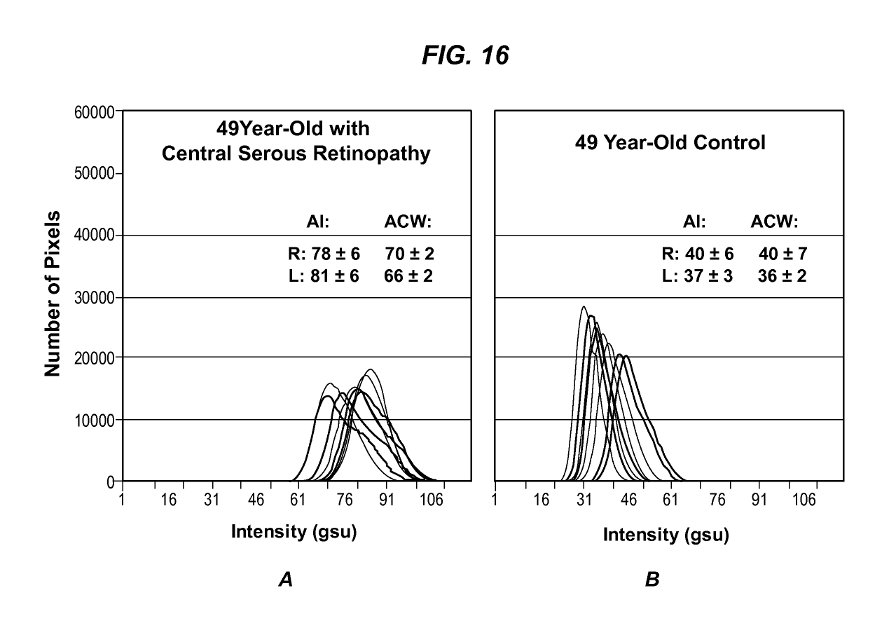

A 49 year-old patient with history of bilateral central serous retinopathy OU was imaged for FA. This patient noted small bilateral paracentral scotomata on Amsler grid testing. VA was 20/20 OU, and fundus examination revealed parafoveal pigment disruption corresponding to the Amsler changes. OCT demonstrated no subretinal or sub-RPE fluid OU, but fluorescein angiography demonstrated areas of mild hyperfluorescence corresponding to the areas of Amsler grid abnormality OU. FA imaging demonstrated a significant increase in AI and ACW in both eyes (AI: OD=78+6 gsu, OS=81+6 gsu; ACW: OD=70+2 gsu, OS=66+2 gsu) compared to an age matched control volunteer (AI: OD=40+6 gsu, OS=37+3 gsu; ACW: OD=40+7 gsu, OS=36+2 gsu). (FIGS. 16A and 16B)

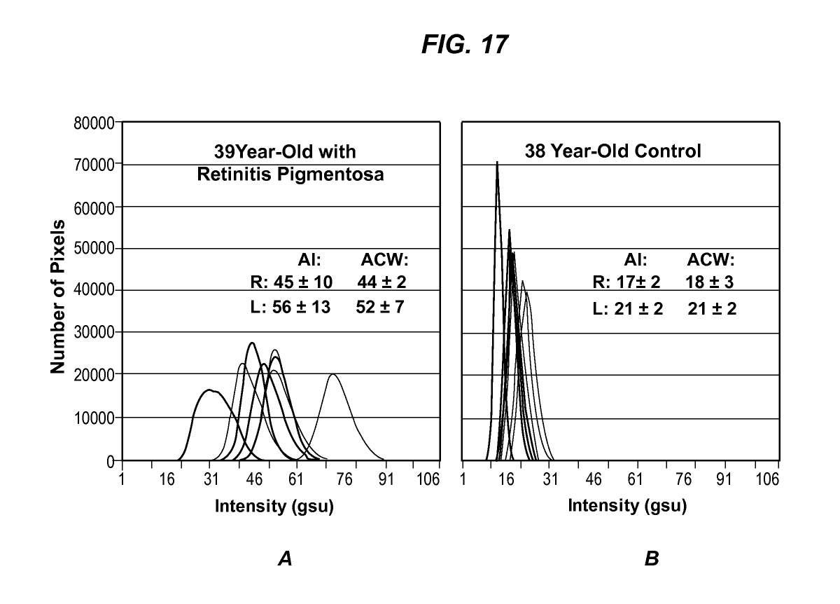

Retinal FA imaging was also performed on a 36 year-old with a diagnosis of retinitis pigmentosa. The patient had VA 20/20 OU, optic disc pallor, retinal vessel attenuation, mild bone-spicule pigmentary deposits, and a ring scotoma on Goldman visual field testing in both eyes. The photopic electroretinogram (ERG) was decreased and the rod-isolated ERG was barely recordable in both eyes. Retinal FA imaging demonstrated a marked increase in the AI and ACW in both eyes (AI: OD=45+10 gsu, OS=56+13 gsu; ACW: OD=44+2 gsu, OS=52+7 gsu) compared to an age-matched control volunteer (AI; OD=17+2 gsu, OS=21+2 gsu; ACW: OD=18+3 gsu, OS=21+2 gsu). (FIGS. 17A and 17B)

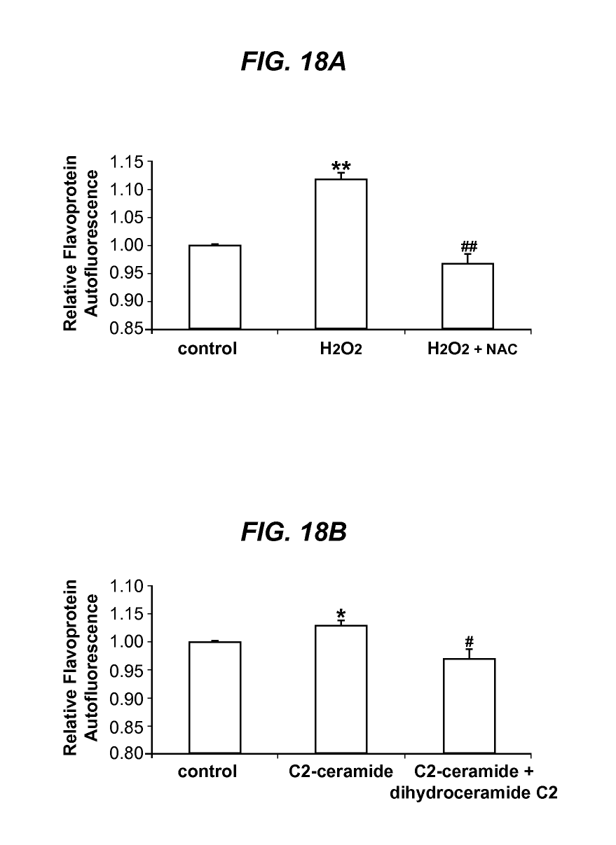

To show that pre-apoptotic conditions induce FA elevations in retinal cells, cultured retinal cells were subjected to hydrogen peroxide and ceramide, both of which are known to induce apoptosis of these cells. As shown in FIGS. 18A and 18B, exposure to each agent for 2 hours resulted in significantly elevated FA. In the case of hydrogen peroxide, the antioxidant, N-acetylcysteine, completely blocked FA elevations. For ceramide, its competitive antagonist, dihydroceramide, also completely blocked FA increases. In fact, the inhibitors tended to reduce FA, compared to control cells, suggesting improved redox status of the cells' mitochondria compared to control cells. As both agents were present only briefly to the pro-apoptotic stimuli, no morphologic evidence of apoptosis by TUNEL or Annexin-V assays was found. These data suggest that as has been observed in cells of the heart, brain, and cartilage, retinal cells under apoptotic conditions exhibit enhanced FA.

### Discussion

FA of human and animal tissues has been known for almost 40 years and has been used study metabolic dysfunction of skeletal muscle, liver, heart, and other tissues. Due to inherent low signal to noise ratios, most descriptions of FA monitoring have been performed on cells and tissues ex vivo using fluorescence microscopy. These studies have shown the dependence of FA fluorescence on the redox status of NAD(P)H and FAD in a reciprocal relationship. Under physiologic conditions in which electron transport is efficient, FAD is found in a reduced state. Reduced FAD molecules in the respiratory chain embedded in the mitochondrial membranes do not fluoresce when exposed to blue light. However, when electron transport is deficient as occurs when the mitochondrial membrane potential is unstable or lost during pro-apoptotic conditions, the FAD molecules become oxidized and their electrons assume maximum resonance becoming prone to brief excitation to a higher energy state by blue light followed by reversion to lower energy orbitals with the emission of green light.

Our results using instrumentation customized to detect FA of retinal cells in vitro demonstrates enhanced signals when HRPE cells are exposed briefly to H2O2 and ceramide at concentrations that have been shown to induce apoptosis. Since our cells were passaged 5 days before experiments were performed it is unlikely that other fluorophores such as lipofuscin, advanced glycated end products (AGEs) or collagen were responsible for the FA signal we detected. Furthermore, when specific inhibitors antagonized the pro-apoptotic effects of H2O2 and ceramide, enhanced FA signal was not detected. These data support the contention that FA oxidation, due to instability of the mitochondrial membrane potential (y) is responsible for the autofluorescence at 535 nm upon excitation with 467 nm light, as performed in our study.

Retinal cell apoptosis is a key pathophysiologic mechanism in many systemic and retinal diseases, including diabetes, age-related macular degeneration, and inherited retinal degenerations. In animal models of diabetic retinopathy, for example hyperglycemia has been shown to lead to oxidative damage of the retinal mitochondria by increased mitochondrial production of superoxide, leading to translocation of pro-apoptotic Bax into mitochondria, activation of capase 3, and apoptosis of retinal pericytes and endothelial cells. These effects have been shown to be mitigated by antioxidants, superoxide dismutase, vitamin E, and alpha-lipoic acid which reduce levels of superoxide produced in the mitochondria, leading to better preservation of retinal capillaries in diabetic rats. Although the initiating insults to retinal cells by systemic and retinal diseases are diverse, affecting either the intrinsic or extrinsic apoptotic pathways, all ultimately effect mitochondrial membrane stability and lead to the opening of the mitochondrial membrane pore causing loss of membrane potential and disruption of electron transport. As an essential component of electron transport, FAD oxidation is an early indication of mitochondrial dysfunction before significant disease-associated cell loss occurs.

Apparatus such as described in U.S. patent application Ser. No. 10/777,423 to Petty et al. now permit detection of FA even in vivo despite the inherent low signal to noise ratio of oxidized FA. In recent in vivo studies on ischemic-reperfusion injury of heart muscle, in experimental animals, FA was shown to increase during reperfusion-induced cellular injury. The FA signal intensity correlated well with measures of apoptosis. Other studies using inhibitors of mitochondrial membrane pore opening have shown that increases in FA signal during pro-apoptotic conditions, is dependent on pore opening leading to FAD oxidation. These studies give strong support to the use of FA as a marker for metabolic instability, if the technical problems of signal to noise ratio and confounding fluorophores can be overcome.

At least some embodiments of the present invention do address these two issues. First, the use of high efficiency photomultiplying CCD chips and photomultiplier detectors, for example, improve the ability to detect the weak signals emitted by oxidized FAD when stimulated by blue light. Secondly, the use of narrowband high-technology filters, for example, enable selection of a small bandwidth at the extreme shoulder of the lipofuscin emission curve, permitting separation of the FA signal from the emission of the dominant retinal fluorophore, lipofuscin. Computer processing by specialized hardware and custom software, for example, permit further enhancement of the FA signal to permit separating control from diseased tissue in vivo with more sensitivity and specificity.

FA imaging of control individuals showed significant age-dependent increases, that were greatest during the fourth to fifth decades of life likely due to ongoing, physiologic apoptosis, but possibly due to some degree of contamination due to the age-dependent increases in lipofuscin which may contribute to the baseline noise in the FA signal we detect. Nevertheless, at each age patients with diabetes or retinal-specific diseases could be distinguished as having metabolic dysfunction, based on higher FA intensity, when compared to age-matched controls. Furthermore, diabetics with retinopathy had significantly higher FA signals than those without retinopathy, indicating that FA may be useful in monitoring the severity of metabolic tissue dysfunction. The higher FA signals in those with retinopathy also make it unlikely that the FA signal is due to the accumulation of lipofuscin, a fluorophore that has not been found to be increased in diabetes. In fact, Hammer and coworkers in a recent paper using a broad excitation band width, found the ratio of green to red fluorescence to be higher in diabetics than controls. As red is the main wavelength emitted by lipofuscin, the authors postulated that lipofuscin was not increased, but that the green signal resulted from “a hint of AGEs” autofluorescence. This is unlikely as the bandwidth used for their in vivo imaging did not include the low wavelength blue/ultraviolet light needed to stimulate AGEs. As previously reported in a study on patients with pseudotumor cerebri, we found that the eyes of patients with systemic or retinal disease reported have also showed considerable asymmetry in FA AI and ACW when compared to control individuals who showed highly symmetric FA values. Thus, FA asymmetry is an important indicator of retinal metabolic dysfunction and is unlikely due to asymmetrical accumulation of other fluorophores. The in vivo data herein taken together with the clear emission of FA by cultured retinal cells supports FAD and not collagen, AGEs, or lipofuscin as the origin of the signal detected.

### Example 5

The following experiment is a flavoprotein fluorescence (FPF) analysis of three unilateral central serous retinopathy (CSR) patients in order to further show the utility of FPF as an indicator of CSR-induced retinal metabolic stress.

CSR is characterized by idiopathic breakdown of the outer blood-retina barrier formed by the retinal pigment epithelium (RPE). The etiology of the disease is unknown, but fluorescein and indocyanine green (ICG) angiographic studies have shown that the pathogenesis involves RPE and choriocapillaris dysfunction, as well as choroidal lobular ischaemia and venous congestion. Optical coherence tomography (OCT) detects neurosensory retinal and RPE detachments, chronic exudates, and cystic changes within the retina. Hypofluorescent or hyperfluorescent fundus autofluorescence is attributed to changes in subretinal and RPE lipofuscin content.

### Materials and Methods

Three men, aged 35, 42, and 30 years, with unilateral CSR and no other ophthalmic or systemic disease underwent retinal FPF analysis after a routine funduscopic examination.

To measure retinal FPF in humans, a fundus camera was modified as previously described with narrow-band excitation and emission filters, a high-sensitivity EMCCD camera, and attached computers with customized software. After pupillary dilation, four 535 nm FPF acquisitions, each induced by a 1 ms, 467 nm incident flash, were obtained over a three degree field from each eye. Due to the instrument's depth of focus FPF was captured from all retinal layers. The images were stored as 512×512 pixel 16-bit grayscale TIFF files. Histogram curves of pixel intensities for each eye were extracted to yield average intensity (AI) of retinal FPF using a method previously described. t test and ANOVA were used to compare AI values between the two eyes of each subject. SAS 9.0 software (SAS Institute Inc., Cary, N.C.) was used for statistical analyses. P-values <0.05 were considered significant.

### Results

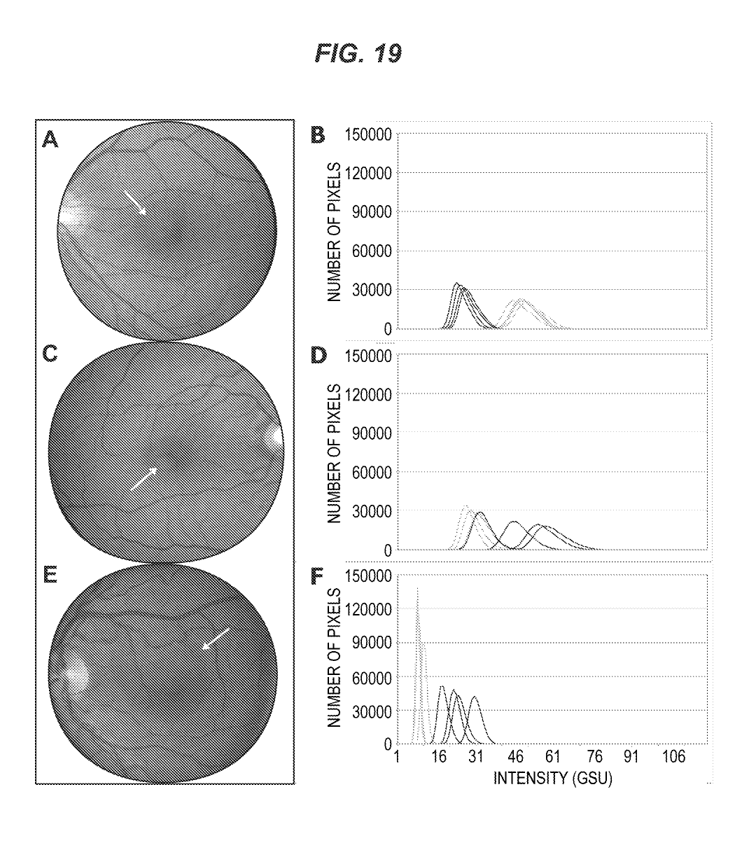

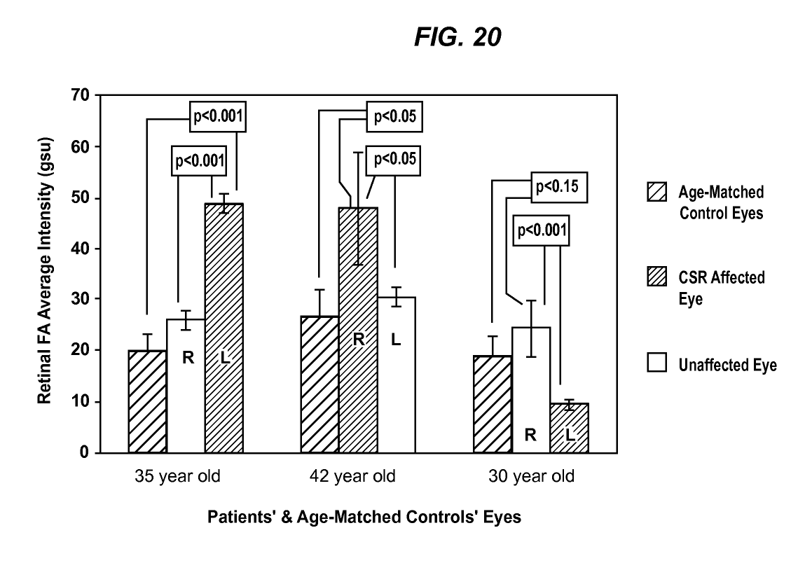

Funduscopic examination, Amsler grid testing, OCT, and fluorescein angiography all showed findings of CSR in each affected eye (FIGS. 19A-19F), whereas the unaffected eyes showed no signs of disease. Present were a PED temporal to the left fovea (19A), a PED inferotemporal to the right fovea (19C), and a blunted foveal reflex with subretinal fluid in the left macula (19E). The histograms showed significantly elevated FPF in each CSR-affected eye compared to the contralateral unaffected eye. Retinal FPF AI of the affected eyes of patient 1 and patient 2 (35 year old and 42 year old), were statistically greater than those of eyes of three age-matched control subjects (p-value: <0.001 and <0.05, respectively). Retinal FPF AI of the affected eye of patient 3 (30 year old) was 30% greater than the eyes of age-matched controls, but did not reach statistical significance (p<0.15) (FIG. 20). Importantly, significant asymmetry existed between the affected eye and the unaffected eye of each CSR patient (p-value: <0.001, <0.05, and <0.001).

### Discussion

Autofluorescence detection of oxidized flavoproteins is distinct from previously used methods to detect fundus autofluorescence, which is primarily due to lipofuscin. As previously described, we minimized the contribution of lipofuscin or lens autofluorescence to our signal by choosing young patients, comparing their affected eye to their unaffected eye, and using a very narrow emission band at the FPF maximum, which excludes most of the emission intensity of lipofuscin, in order to obtain maximal metabolic contrast.

Conventional fundus autofluorescence, induced by 488 nm light, shows hypofluorescence at the focal sites of leakage several weeks after onset in CSR-affected eyes. The hypofluorescence is presumably due to either reduced lipofuscin from mechanical disruption of the RPE, or blockage of RPE lipofuscin by accumulating subretinal fluid. Some reports in acute and chronic CSR describe hypofluorescence and hyperfluorescence surrounding the original point of leakage; hyperfluorescence is attributed to accumulation of lipofuscin pigment in surviving RPE cells, while hypofluorescence in chronic CSR lesions is thought to result from reduced RPE metabolic activity as photoreceptors are lost or from progressive RPE cell loss.

Retinal FPF AI was significantly elevated in each CSR-affected eye, regardless of disease duration, when compared to the FPF AI of the contralateral unaffected eye. It was also greater than in age-matched control eyes. The ability to detect elevated FPF in CSR as early as one week after disease onset contrasts with alterations in conventional fundus autofluorescence that do not develop until several weeks after disease onset, indicating that our FPF signal is probably not due to lipofuscin, but is the result of impaired mitochondrial metabolic activity. Thus, FPF may be beneficial in the early diagnosis of CSR when retinal metabolic activity is compromised, but before substantial cell loss, presumably from apoptosis, occurs.

This study demonstrated two characteristics of FPF that indicate disease: 1) elevated retinal FPF AI in a patient's eye compared to that of age-matched control eyes (FIG. 20), and 2) significantly increased retinal FPF AI of a patient's eye compared to that of their contralateral eye (FIGS. 19A-F and 20). This asymmetry is not present in any age-matched control volunteer, indicating that asymmetry between patients' eyes is a characteristic of disease, as was previously shown for pseudotumor cerebri. This can be true even when retinal FPF AI of an eye is not statistically different from the retinal FPF AI of eyes of age-matched control volunteers (FIG. 20), as in patient 3. This study suggests that rapid, non-invasive FPF analysis of the human retina is feasible for detecting CSR-induced retinal dysfunction.

### Example 6

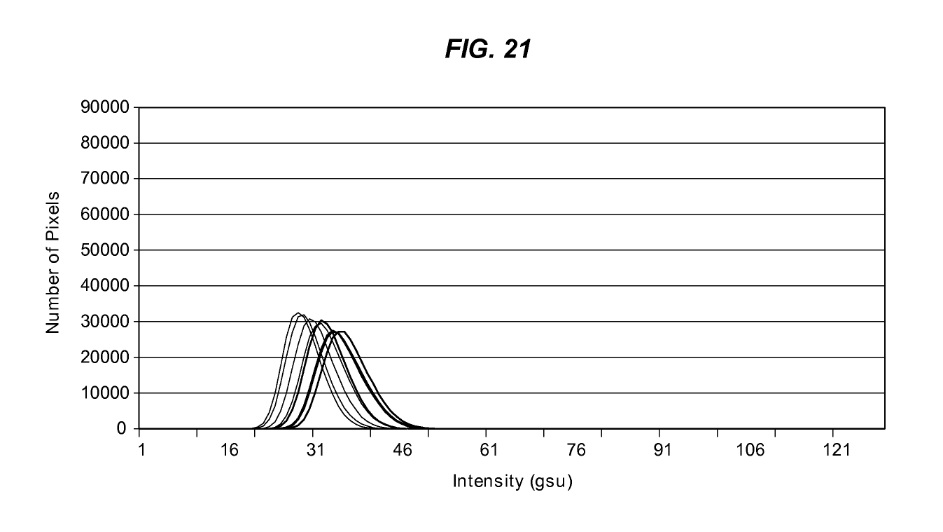

Studies with two glaucoma patients and controls were performed. FIG. 21 shows a control study. For each measurement, the intensity of each pixel (in grey scale units, gsu) is plotted at the abscissa whereas the number of pixels with that intensity is given at the ordinate. The patient's right eye is blue and the left eye is red. To illustrate the reproducibility of the measurements, multiple graphs of the same eye are overlaid in each diagram. Although there is some variability in the measurements, the findings are highly reproducible.

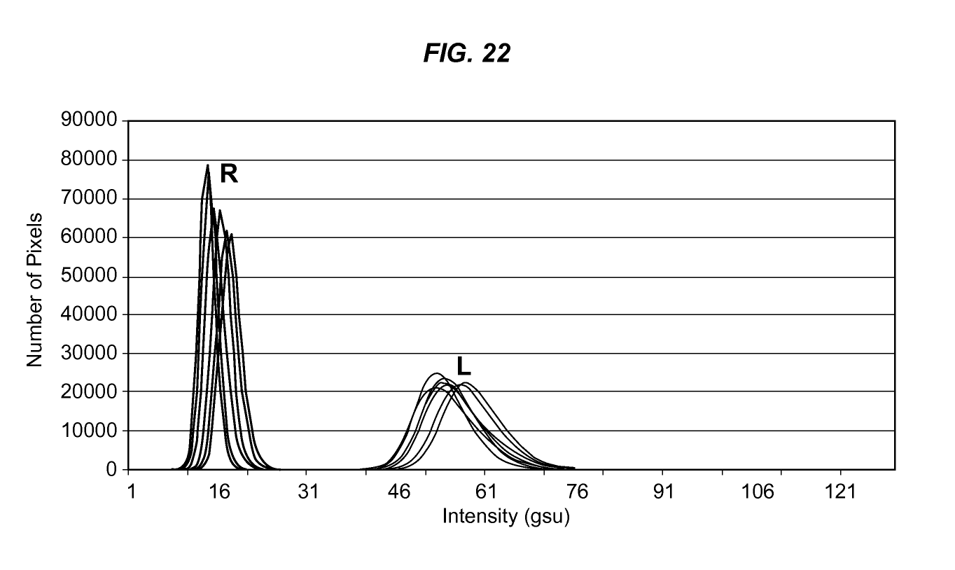

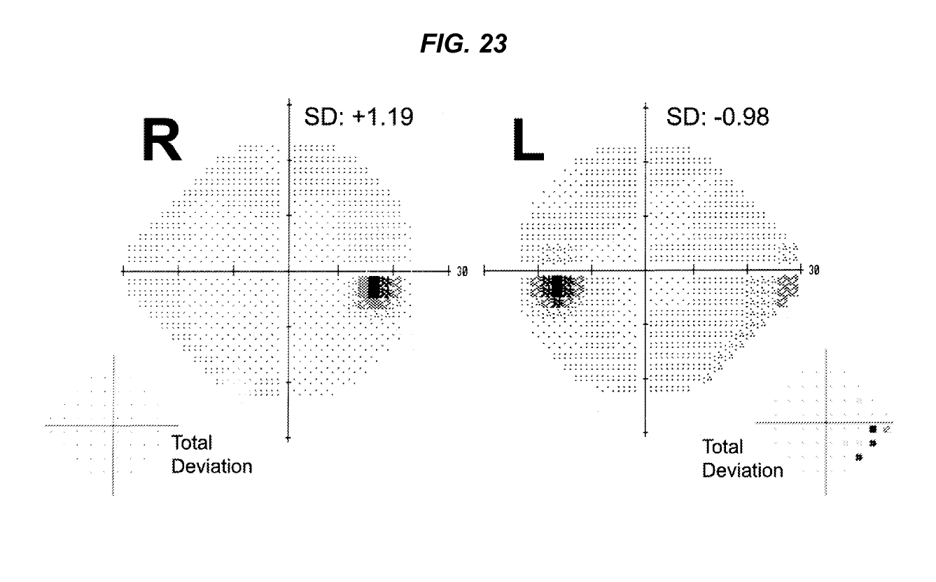

FIGS. 22 and 23 show data from a glaucoma patient. This particular patient was chosen because his left eye was more affected by disease. This is supported by the clinical findings as well as imaging studies that clearly indicated that the amount of optic disc cupping was much greater in the left eye than the right eye (data not shown). This finding was also consistent with visual field testing (FIG. 23). The asymmetry provides us with an internal control for the left eye. That is, it is identical to the right (color, age, sex, etc.) except for the extent of disease. In this case, the more affected eye displayed an average intensity of 55.3±1.6 gsu and a curve width of 50.6±1.5 gsu whereas the values of the less affected eye were 16.4±1.6 gsu and 17.4±3.4, respectively. Thus, changes were detected in autofluorescence intensity and curve width associated with metabolism in this glaucoma patient. Moreover, the subtle visual field defect (FIG. 23) is accompanied by robust FA differences (FIG. 22) because FA detects earlier changes in the neural retina. Similar results were obtained using another glaucoma patient with asymmetric disease.

### Example 7

To provide an in vitro correlate of the imaging method described above, studies were pursued of: RPE cells in vitro and fresh pieces of rat and human retina ex vivo. Both retinal fragments and RPE cells serve as good in vitro models of the human retina. To model the apoptotic events taking place in the eye, apoptotic stimuli (H2O2 and ceramide) were added to these samples. These stimuli are particularly judicious choices because both oxidative molecules and ceramide have been reported to be key mediators of retinal apoptosis. Hence, these studies form a good in vitro correlate of the events occurring in many blinding eye diseases.

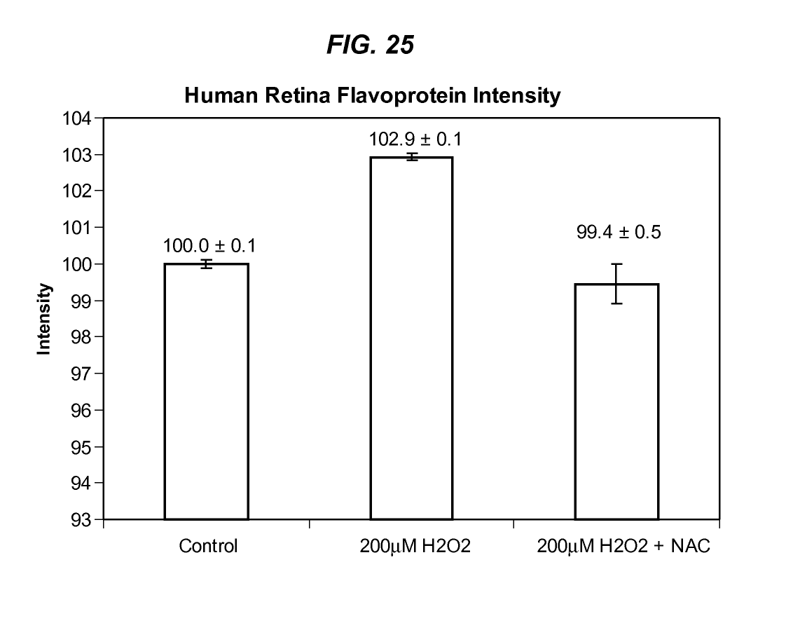

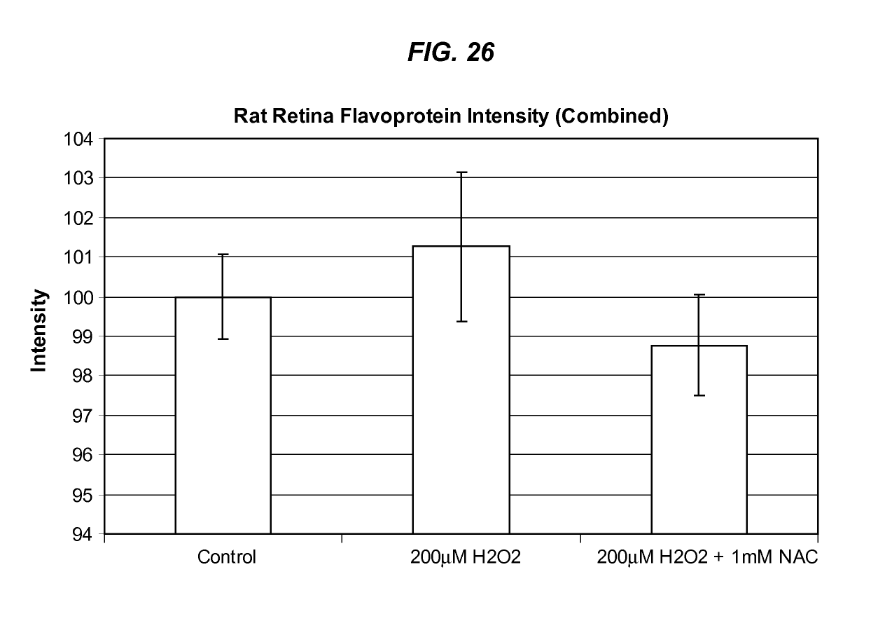

RPE cells and fresh retina pieces from humans and rats exhibit elevated flavoprotein autofluorescence (FA) when subjected to sublethal or lethal concentrations of apoptotic stimuli (H2O2 and ceramide) (FIGS. 24-26 and 18A-18B). At lethal levels, FA correlates with apoptosis measured by TUNEL and other assays.

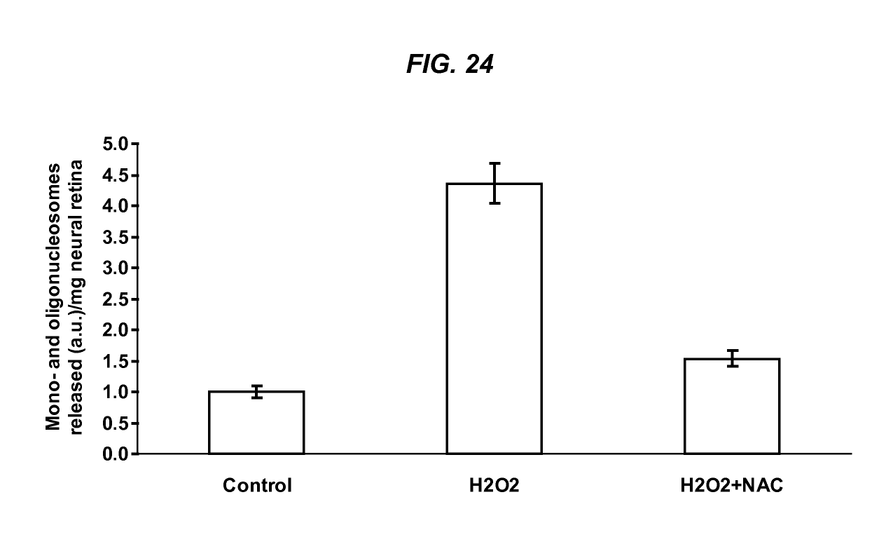

To evaluate neural retinal apoptosis, pieces of human neural retina were analyzed by using a Cell Death Detection ELISAPLUS kit. This photometric enzyme immunoassay provides the quantitative in vitro determination of cytoplasmic histone-associated DNA fragments (mono- and oligonucleosomes) after induced cell death (shown in FIG. 24). Cell death was increased when the cells were induced by hydrogen peroxide and cell death by hydrogen peroxide was attenuated with the presence of the oxidation inhibitor N-acetyl-cysteine. Data is shown in TABLES 2 and 3 below.

TABLE 2
6 hr data from human
neural retina

| | | | | | P | P |
|---|---|---|---|---|---|---|
| | | | | | value | value |
| | | | | | vs. | vs. |
| H₂O₂ (M) | | avarage | sem | n | control | H₂O₂ |
| 0 | Control | 1.0000 | 0.1019 | 4 | P < | |
| | | | | | 0.001 | |
| 200 | H₂O₂ | 4.3610 | 0.3171 | 3 | P > | P < |
| | | | | | 0.05 | 0.001 |
| 200 + 1 mM | H₂O₂ + NAC | 1.5362 | 0.1219 | 3 | | |
| NAC | | | | | | |

Note:
n = assay replicates
Neural retinal apoptosis was estimated with Cell Death Detection ELISA.

TABLE 3

| | |
|---|---|
| p-value between control and 200 μM H2O2 = | 0.03 |
| p-value between control and 200 μM H2O2 + NAC = | 0.007 |
| p-value between 200 μM H2O2 and 200 μM H2O2 + NAC = | <0.001 |

n = 6 retinas, 14-16 acquisitions

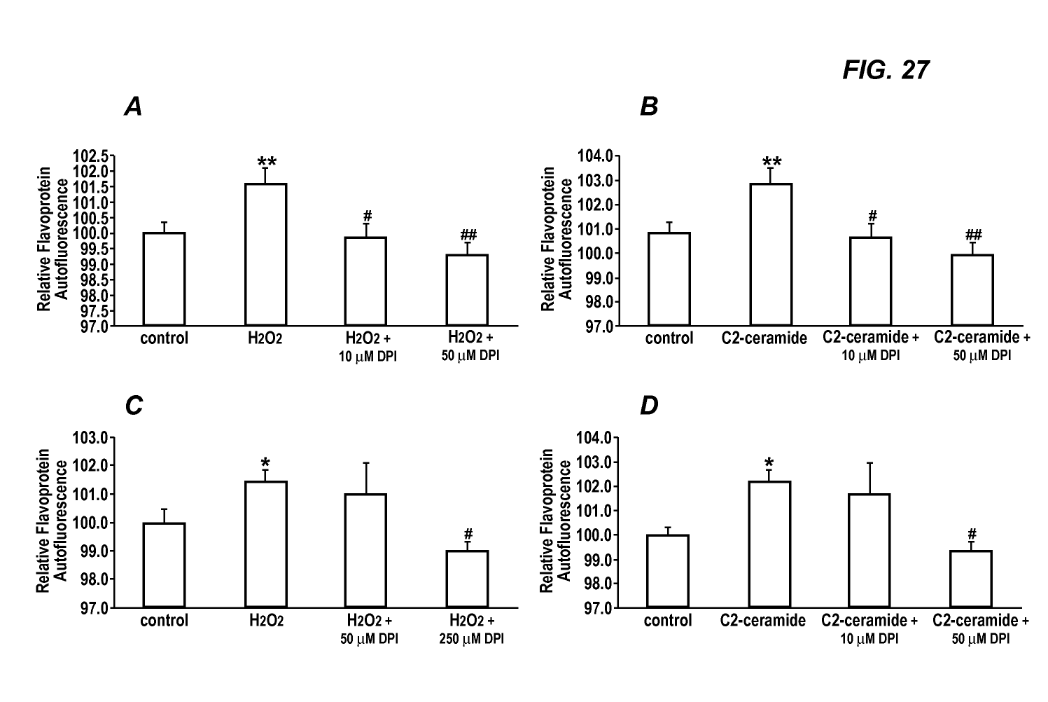

Specific toxins (DPI and NaCN) abrogate the FA signal, indicating its metabolic origin as opposed to other fixed fluorophores such as luteins, lipofuscin, or AGEs (FIG. 27, P values: *P<0.05, **P<0.001, compared with control; #P<0.05, ##P<0.01, compared with H2O2- or C2-ceramide-stimulated cells.). These data show that during apoptotic conditions retinal cells exhibit enhanced FA, which is consistent with studies of other cell types.

The data disclose novel applications for embodiments of devices and derivative devices according to the present invention whose applications include, but are not limited to detection of, monitoring of, and/or monitoring treatment of: diabetes, pre-diabetes, diabetic retinopathy, age-related macular degeneration, glaucoma, retinal vascular disease, infectious and non-infectious uveitis and retinitis, cancer, inflammation, inherited retinal degenerations, central serous retinopathy and other acquired retinopathies, pseudotumor cerebri, Graves' disease, optic neuritis (multiple sclerosis), etc. The findings for diabetes are especially important as over one-half of the US population will be diabetic or pre-diabetic by the year 2018, according to CDC statistics.

Several specific applications of retinal autofluorescence imaging were presented above, but it is evident to those skilled in the art that the applications are not limited to the details of the foregoing illustrative examples and include modifications for use on other ocular tissues, including the cornea and lens, in animals and humans. Any application expressing changes in retinal or other ocular tissue stress due to ocular or systemic dysfunction induced by disease or agents would be detectable along the lines we have presented. Included, for example, are factors such as hypertension and agents (including drugs) with toxic effects. Applications for monitoring disease progression and mitigation by treatments (including drugs) on humans and animals or their tissues or cells is included, as well as treatments aimed at eliminating unwanted cell populations as in cancer or inflammation. Similarly, patients, animals, and their tissues and cells can be monitored for drug effects. This approach can also be applied in other settings such as drug discovery.

Throughout this application, various publications, including United States patents and patent applications, are referenced by author and year and patents by number. The disclosures of these publications and patents in their entireties are hereby incorporated by reference into this application in order to more fully describe the state of the art to which this invention pertains.

The invention has been described in an illustrative manner, and it is to be understood that the terminology which has been used is intended to be in the nature of words of description rather than of limitation.

While the present invention has been described with reference to specific examples, which are intended to be illustrative only and not to be limiting of the invention, it will be apparent to those of ordinary skill in the art that changes, additions and/or deletions may be made to the disclosed embodiments without departing from the spirit and scope of the invention.

What is claimed is:

1. A method comprising:
measuring, in vivo and non-invasively, ocular flavoprotein fluorescence in ocular cells or ocular tissue with impaired electron transport or mitochondrial membrane instability; and
determining from the measured ocular flavoprotein fluorescence ocular cells or ocular tissue that are in a pre-apoptotic state, wherein the level of ocular flavoprotein fluorescence indicates the functional status of mitochondria.

2. The method of claim 1, further comprising determining from the ocular flavoprotein fluorescence if the ocular cells or ocular tissue in the pre-apoptotic state are prone to apoptosis and undergo apoptosis.

3. The method of claim 1, further comprising determining from the ocular flavoprotein fluorescence if the ocular cells or ocular tissue in the pre-apoptotic state are prone to apoptosis but are rescued by endogenous intervention.

4. The method of claim 1, further comprising determining from the ocular flavoprotein fluorescence if the ocular cells or ocular tissue in the pre-apoptotic state are prone to apoptosis but are rescued by exogenous intervention, including a medical therapy.

5. The method of claim 1, further comprising assessing the progression of future disease based on ocular flavoprotein fluorescence.

6. The method of claim 1, further comprising assessing (1) the pre-apoptotic state; and/or (2) the risk for disease progression based on ocular flavoprotein fluorescence in combination with other demographic and medical information from a human or animal subject.

7. A method comprising:
measuring, in vivo and non-invasively, flavoprotein fluorescence in any cell or tissue type with impaired electron transport or mitochondrial membrane instability; and
determining from the measured flavoprotein fluorescence cells or tissue that are in a pre-apoptotic state, wherein the level of flavoprotein fluorescence indicates the functional status of mitochondria.

8. The method of claim 7, further comprising determining from the flavoprotein fluorescence if the cells or tissue in the pre-apoptotic state are prone to apoptosis and undergo apoptosis.

9. The method of claim 7, further comprising determining from the flavoprotein fluorescence if the cells or tissue in the pre-apoptotic state are prone to apoptosis but are rescued by endogenous intervention.

10. The method of claim 7, further comprising determining from the flavoprotein fluorescence if the cells or tissue in the pre-apoptotic state are prone to apoptosis but are rescued by exogenous intervention, including a medical therapy.

11. The method of claim 7, further comprising assessing the progression of future disease based on flavoprotein fluorescence of the cells or tissue.

12. The method of claim 7, further comprising assessing (1) the pre-apoptotic state; and/or (2) the risk for disease progression based on flavoprotein fluorescence in combination with other demographic and medical information from a human or animal subject.
